(19) United States
(12) Patent Application Publication
(10) Pub. No.: US 2002/0072521 A1
(43) Pub. Date: Jun. 13, 2002

(54) 5-substituted arylpyrimidines

(76) Inventors: Taeyoung Yoon, Guilford, CT (US); Stephane DeLombaert, Madison, CT (US); Kevin J. Hodgetts, Killingworth, CT (US); Dario Doller, Wallingford, CT (US)

Correspondence Address: EDWARDS & ANGELL, LLP, Dike, Bronstein, Roberts & Cushman, Intellectual Property Patent Practice, P.O. Box 9169, Boston, MA, 02109, US

(21) Appl. No.: 09/811,359

(22) Filed: Mar. 16, 2001

Related U.S. Application Data
(60) Provisional application No. 60/189,774, filed on Mar. 16, 2000.
(60) Provisional application No. 60/206,454, filed on May 22, 2000.

(51) Int. Cl. A61K 31/5377; A61K 31/506; A61K 31/505; C07D 413/04; C 07D 4 3/04; C07D 239/02

(52) U.S. Cl. 514/235.8; 514/252.14; 514/275; 544/295; 544/323; 544/122

(57) ABSTRACT

Arylpyrimidine compounds are provided that can act as selective modulators of CRF receptors. These compounds are useful in the treatment of a number of CNS and periphereal disorders, particularly stress, anxiety, depression, cardiovascular disorders, and eating disorders. Methods of treatment of such disorders and well as packaged pharmaceutical compositions are also provided.

Compounds of the invention are also useful as probes for the localization of CRF receptors and as standards in assays for CRF receptor binding. Methods of using the compounds in receptor localization studies are given.

This application claims the benefit of U.S. Provisional Application Ser. No. 60/189,774 filed Mar. 16, 2000 and of U.S. Provisional Application Ser. No. 60/206,454 filed May 22, 2000, the teachings of which are incorporated herein by reference.

### FIELD OF THE INVENTION

The present invention relates to novel substituted arylpyrimidine compounds that bind with high selectivity and/ or high affinity to CRF1 receptors (Corticotropin Releasing Factor 1 Receptors). This invention also relates to pharmaceutical compositions comprising such p compounds and to the use of such compounds in treatment of psychiatric disorders and neurological diseases, including major depression, anxiety-related disorders, post-traumatic stress disorder, supranuclear palsy and feeding disorders, as well as treatment of immunological, cardiovascular or heart-related diseases and colonic hypersensitivity associated with psychopathological disturbance and stress. Additionally this invention relates to the use such compounds as probes for the localization of CRF1 receptors in cells and tissues.

### BACKGROUND OF THE INVENTION

Corticotropin releasing factor (CRF), a 41 amino acid peptide, is the primary physiological regulator of proopiomelanocortin (POMC) derived peptide secretion from the anterior pituitary gland. In addition to its endocrine role at the pituitary gland, immunohistochemical localization of CRF has demonstrated that the hormone has a broad extrahypothalamic distribution in the central nervous system and produces a wide spectrum of autonomic, electrophysiological and behavioral effects consistent with a neurotransmitter or neuromodulator role in brain. There is also evidence that CRF plays a significant role in integrating the response of the immune system to physiological, psychological, and immunological stressors.

Clinical data provide evidence that CRF has a role in psychiatric disorders and neurological diseases including depression, anxiety-related disorders and feeding disorders. A role for CRF has also been postulated in the etiology and pathophysiology of Alzheimers disease, Parkinsons disease, Huntingtons disease, progressive supranuclear palsy and amyotrophic lateral sclerosis as they relate to the dysfunction of CRF neurons in the central nervous system.

In affective disorder, or major depression, the concentration of CRF is significantly increased in the cerebral spinal fluid (CSF) of drug-free individuals. Furthermore, the density of CRF receptors is significantly decreased in the frontal cortex of suicide victims, consistent with a hypersecretion of CRF. In addition, there is a blunted adrenocorticotropin (ACTH) response to CRF (i.v. administered) observed in depressed patients. Preclinical studies in rats and non-human primates provide additional support for the hypothesis that hypersecretion of CRF may be involved in the symptoms seen in human depression. There is also preliminary evidence that tricyclic antidepressants can alter CRF levels and thus modulate the numbers of CRF receptors in brain.

CRF has also been implicated in the etiology of anxiety-related disorders. CRF produces anxiogenic effects in animals and interactions between benzodiazepine/non-benzodiazepine anxiolytics and CRF have been demonstrated in a variety of behavioral anxiety models. Preliminary studies using the putative CRF receptor antagonist (α-helical ovine CRF (9-41) in a variety of behavioral paradigms demonstrate that the antagonist produces “anxiolytic-like” effects that are qualitatively similar to the benzodiazepines. Neurochemical, endocrine and receptor binding studies have all demonstrated interactions between CRF and benzodiazepine anxiolytics providing further evidence for the involvement of CRF in these disorders. Chlordiazepoxide attenuates the anxiogenic effects of CRF in both the conflict test and in the acoustic startle test in rats. The benzodiazepine receptor antagonist Ro 15-1788, which was without behavioral activity alone in the operant conflict test, reversed the effects of CRF in a dose-dependent manner, while the benzodiazepine inverse agonist FG 7142 enhanced the actions of CRF.

CRF has also been implicated in the pathogeneisis of certain immunological, cardiovascular or heart-related diseases such as hypertension, tachycardia and congestive heart failure, stroke and osteoporosis, as well as in premature birth, psychosocial dwarfism, stress-induced fever, ulcer, diarrhea, post-operative ileus and colonic hypersensitivity associated with psychopathological disturbance and stress.

The mechanisms and sites of action through which conventional anxiolytics and antidepressants produce their therapeutic effects remain to be fully elucidated. It has been hypothesized however, that they are involved in the suppression of CRF hypersecretion that is observed in these disorders. Of particular interest are that preliminary studies examining the effects of a CRF receptor antagonist peptide (α-helical CRF₉₋₄₁) in a variety of behavioral paradigms have demonstrated that the CRF antagonist produces anxiolytic-like effects qualitatively similar to the benzodiazepines.

### Description of the Related Art

A number of small molecule compounds for the treatment of CRF related disorders have been disclosed in the literature [for a review see J. McCarthy et al. Curr. Pharm. Des. 5: 289(1999)].

McCarthy et al. (WO 96/39400) have disclosed aryl pyrimidine derivatives of the general formula

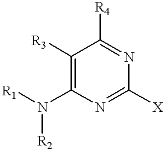

wherein X, R₁, R₂, R₃, and R₄ are defined therein, for use as CRF receptor in the treatment of central nervous system disorders. The McCarthy application discloses arylpyrimidine compounds that contain a disubstituted amino group (NR₁R₂) in the 4-position of the pyrimidine ring. It is therefore surprising that the novel pyrimidines of this invention, which lack the corresponding disubstituted NR₁R₂ group in the 4-position of the pyrimidine ring, are also CRF receptor antagonists.

### SUMMARY OF THE INVENTION

The invention provides novel compounds of Formula I (shown below), and pharmaceutical compositions comprising compounds of Formula I and at least one pharmaceutically acceptable carrier or excipient. Such arylpyrimidines bind to cell surface receptors, preferably G-coupled protein receptors, especially CRF receptors and most preferably CRF1 receptors. Preferred compounds of the invention exhibit high affinity for CRF1 receptors.

Additionally, preferred compounds of the invention also exhibit high specificity for CRF1 receptors.

Typically preferred compounds of the invention include those of Formula I:

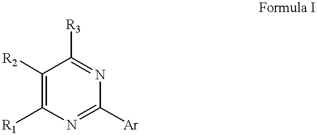

and the pharmaceutically acceptable salts thereof, wherein:

Ar is phenyl, 1- or 2-naphthyl, each of which is mono-, di-, or tri-substituted or mono-, di-, or tri-substituted heteroaryl having from about 5 to about 7 ring members and 1 to about 4 heteroatoms in the ring, the heteroatoms independently selected from the group consisting of N, O and S;

R₁ and R₃ are independently chosen from hydrogen, halogen, cyano, nitro, optionally substituted alkyl, optionally substituted alkenyl, optionally substituted alkynyl, optionally substituted alkoxy, optionally substituted (cycloalkyl)alkyl, optionally substituted alkylthio, optionally substituted alkylsulfinyl, optionally substituted alkylsulfonyl, or optionally substituted mono-or dialkylcarboxamide, with the proviso that R₁ and R₃ are not both hydrogen; and

R₂ is optionally substituted alkyl, optionally substituted alkenyl, optionally substituted alkynyl, optionally substituted alkoxy, optionally substituted aminoalkyl, optionally substituted mono or dialkylamino, optionally substituted alkylthio, optionally substituted alkylsulfinyl, optionally substituted alkylsulfonyl, optionally substituted mono or dialkylcarboxamide, optionally substituted carbocyclic aryl or optionally substituted heteroaryl having from 1 to 3 rings, and 3 to 8 ring members in each ring and 1 to about 3 heteroatoms.

Particular embodiments of this invention include compounds of Formula I in which R₁ and R₃ are as defined above for Formula I, Ar is phenyl which is mono-, di-, or tri-substituted; and

R₂ is selected from optionally substituted alkyl, optionally substituted alkoxy, optionally substituted aminoalkyl, optionally substituted mono or dialkylamino, optionally substituted alkylthio, optionally substituted alkylsulfinyl, optionally substituted alkylsulfonyl, optionally substituted mono and dialkylcarboxamide, or

R₂ is selected from the group consisting of phenyl, naphthyl, pyridyl, pyrimidinyl, pyridizinyl, and thiophenyl, each of which is optionally mono-, di-, or tri-substituted.

Particular embodiments of this invention also include compounds of Formula I in which R₁ and R₃ are as defined above for Formula I, Ar is mono-, di-, or trisubstituted phenyl; and R₂ is selected from optionally substituted alkoxy, optionally substituted aminoalkyl, and optionally substituted mono or dialkylamino.

The invention further comprises methods of treating patients suffering from certain disorders with an effective amount of a compound of the invention. These disorders include CNS disorders, particularly affective disorders, anxiety disorders, stress-related disorders, eating disorders and substance abuse. The patient suffering from these disorders may be a human or other animal (preferably a mammal), such as a domesticated companion animal (pet) or a livestock animal.

According to yet another aspect, the present invention provides pharmaceutical compositions comprising compounds of Formula I or the pharmaceutically acceptable salts or solvates thereof, which compositions are useful for the treatment of the above-recited disorders. The invention further provides methods of treating patients suffering from any of the above-recited disorders with an effective amount of a compound or composition of the invention.

Additionally this invention relates to the use of the compounds of the invention (particularly labeled compounds of this invention) as probes for the localization of receptors in cells and tissues and as standards and reagents for use in determining the receptor-binding characteristics of test compounds.

Preferred arylpyrimidines of the invention exhibit good activity in standard in vitro receptor binding assays, specifically the assay as specified in Example 96, which follows and is defined below. Particularly preferred arylpyrimidines of the invention have an IC₅₀ of about 1 micromolar or less, still more preferably an IC₅₀ of about 100 nanomolar or less even more preferably an IC₅₀ of about 10 nanomolar or less or even 1 nanomolar or less in such a defined standard in vitro CRF receptor binding assay as exemplified by Example 96 which follows.

### DETAILED DESCRIPTION OF THE INVENTION

In addition to compounds of Formula I, shown and described above, the invention also provides compounds and the pharmaceutically acceptable salts of Formula I, which will be referred to as compounds of Formula Ia in which:

R₁ and R₃ are independently selected from hydrogen, halogen, cyano, C₁₋₆ alkyl₁, (C₃₋₇cycloalkyl₁)C₁₋₄alkyl₁, —O(C₃₋₇cycloalkyl₁)C₁₋₄alkyl₁, halo(C₁₋₆)alkyl₁, —O(halo(C₁₋₆) alkyl₁), —O(C₁₋₆alkyl₁), and S(O)ₙ(C₁₋₆alkyl₁),

where each alkyl₁ is independently straight, branched, or cyclic, may contain 1 or more double or triple bonds, and is optionally substituted with one or more substituents independently chosen from halogen, hydroxy, oxo, cyano, C₁₋₄alkoxy, amino, and mono- or di(C₁₋₄)alkylamino, and where each C₃₋₇cycloalkyl₁ is optionally substituted by one or more substituents independently chosen from halogen, hydroxy, oxo, cyano, C₁₋₄alkoxy, amino, and mono- or di(C₁₋₄)alkylamino,

with the proviso that not both R₁ and R₃ are hydrogen;

R₂ is selected from the group consisting of −XR_{A} and Y; and

Ar is selected from the group consisting of phenyl, naphthyl, pyridyl, pyrimidinyl, pyridizinyl, and thiophenyl, each of which is mono-, di-, or tri-substituted with R_{C};

R_{A} and R_{B}, which may be the same or different, are independently selected at each occurrence from: hydrogen and straight, branched, or cyclic alkyl groups, including (cycloalkyl)alkyl groups consisting of 1 to 8 carbon atoms, which straight, branched, or cyclic alkyl groups may contain one or more double or triple bonds, each of which 1 to 8 carbon atoms may be further substituted with one or more substituent(s) independently selected from oxo, hydroxy, halogen, cyano, amino, C₁₋₆alkoxy, —NH(C₁₋₆alkyl), —N(C₁₋₆alkyl)( C₁₋₆alkyl), —NHC(═O) (C₁₋₆alkyl), —N(C₁₋₆alkyl)C(═O)(C₁₋₆alkyl), —NHS(O)ₙ(C₁₋₆alkyl), —S(O)ₙ(C₁₋₆alkyl), —S(O)ₙNH(C₁₋₆alkyl), —S(O)ₙN(C₁₋₆alkyl)(C₁₋₆alkyl), and Z;

R_{C} is independently selected at each occurrence from halogen, cyano, halo(C₁₋₆)alkyl, halo(C₁₋₆)alkoxy, hydroxy, amino, C₁₋₆alkyl substituted with 0-2 R_{D}, C₂₋₆alkenyl substituted with 0-2 R_{D}, C₂₋₆alkynyl substituted with 0-2 R_{D}, C₃₋₇cycloalkyl substituted with 0-2 R_{D}, (C₃₋₇cycloalkyl) C₁₋₄alkyl substituted with 0-2 R_{D}, C₁₋₆alkoxy substituted with 0-2 R_{D}, —NH(C₁₋₆alkyl) substituted with 0-2 R_{D}, —N(C₁₋₆alkyl)(C₁₋₆alkyl) each C₁₋₆alkyl independently substituted with 0-2 R_{D}, −XR_{A}, and Y;

R_{D} is independently selected at each occurrence from the group consisting of halogen, hydroxy, cyano, amino, C₁₋₄alkyl, —O(C₁₋₄alkyl), —NH(C₁₋₄alkyl), —N(C₁₋₄alkyl)(C₁₋₄alkyl), —S(O)ₙ(alkyl), halo(C₁₋₄)alkyl, halo(C₁₋₄)alkoxy, CO(C₁₋₄alkyl), CONH(C₁₋₄alkyl), CON(C₁₋₄alkyl)(C₁₋₄alkyl), −XR_{A}, and Y;

X is independently selected at each occurrence from the group consisting of —CH₂—, —CHR_{B}—, —O—, —C(═O)—, —C(═O)O—, —S(O)ₙ—, —NH—, —NR_{B}—, —C(═O)NH—, —C(═O)NR_{B}—, —S(O)ₙNH—, —S(O)ₙNR_{B}—, —OC(═S)S—, —NHC(═O)—, —NR_{B}C(═O)—, —NHS(O)ₙ—, —OSiHₙ(C₁₋₄alkyl₂₋ₙ—, and —NR_{B}S(O)ₙ—;

Y and Z are independently selected at each occurrence from: 3- to 7-membered carbocyclic or heterocyclic groups which are saturated, unsaturated, or aromatic, which may be further substituted with one or more substituents independently selected from halogen, oxo, hydroxy, amino, cyano, C₁₋₄alkyl, —O(C₁₋₄alkyl), —NH(C₁₋₄alkyl), —N(C₁₋₄alkyl)(C₁₋₄alkyl),and —S(O)ₙ(alkyl),

said 3- to 7-membered heterocyclic groups containing one or more heteroatom(s) independently selected from N, O, and S, with the point of attachment being either carbon or nitrogen; and

n is independently selected at each occurrence from 0, 1, and 2.

Particular embodiments of the invention include compounds and salts of Formula Ia in which Ar is phenyl, mono-, di-, or tri-substituted with R_{C} and R₁, R₂, and R₃ are as defined for Formula Ia.

The invention further includes compounds and salts of Formula Ia in which Ar is phenyl mono-, di-, or tri-substituted with R_{C}; R₁ and R₃ are independently selected groups (1) halogen and (2) C₁₋₃alkyl, C₁₋₃alkoxy, (C₃₋₇cycloalkyl)C₁₋₃alkyl, (C₃₋₇cycloalkyl) C₁₋₃alkoxy, where each member of group (2) is unsubstituted or substituted by 1-3 groups independently chosen from hydroxy, amino, cyano, and halogen.

Also provided by the invention are compounds and salts of Formula Ia in which Ar is phenyl mono-, di-, or tri-substituted with R_{C}; and R_{A} and R_{B}, which may be the same or different, are independently selected at each occurrence from straight, branched, or cyclic alkyl groups having from 1 to 8 carbon atoms, which alkyl groups may contain one or more double or triple bonds.

Further provided as embodiments of the invention are compounds and salts of Formula Ia in which Ar is phenyl mono-, di-, or tri-substituted with R_{C}; R_{A} and R_{B}, which may be the same or different, are independently selected at each occurrence from straight, branched, or cyclic alkyl groups having from 1 to 8 carbon atoms, which alkyl groups may contain one or more double or triple bonds; and R₁ and R₃ are independently selected from groups (1) halogen and (2) C₁₋₃alkyl, C₁₋₃alkoxy, (C₃₋₇cycloalkyl)C₁₋₃alkyl, (C₃₋₇cycloalkyl) C₁₋₃alkoxy, where each member of group (2) is unsubstituted or substituted by 1-3 groups independently chosen from hydroxy, amino, cyano, and halogen.

Also provided as embodiments of the invention are compounds and salts of Formula Ia having the formula

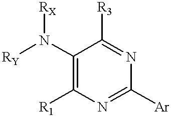

wherein Ar, R₁ and R₃ are as defined for Formula Ia, and R_{X} and R_{Y} are the same or different and are independently selected from hydrogen and C₁-C₆ alkyl; or NR_{X}R_{Y} represents Formula Ib:

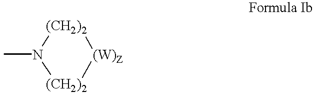

wherein z is 0 or 1; and W is CR_{A}R_{B}, NR_{B}, or O.

A preferred embodiment of the invention includes compounds of Formula A

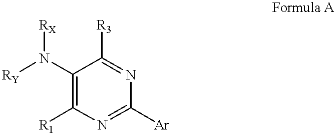

and the pharmaceutically acceptable salt thereof, wherein:

R_{X} and R_{Y} are the same or different and are independently selected from:

a) hydrogen,

b) —(C═O)alkyl_{A}, wherein alkyl_{A} is a straight or branched alkyl group having from 1 to 8 carbon atoms;

c) straight, branched, or cyclic alkyl groups, including cycloalkyl(alkyl)groups, said alkyl groups having from 1 to 8 carbon atoms and optionally containing one or more double or triple bonds, each of which alkyl groups may be further substituted with one or more substituent(s) independently selected from:

i) hydroxy, halogen, amino, cyano, —O(C₁₋₄alkyl), —NH(C₁₋₄alkyl), and —NH(C₁₋₄alkyl)(C₁₋₄alkyl), and

ii) 3- to 7-membered carbocyclic and heterocyclic groups, which are saturated, unsaturated, or aromatic, which may be substituted with one or more substituents independently selected from halogen, halo(C₁₋₄)alkyl, halo(C₁₋₄)alkoxy, oxo, hydroxy, amino, C₁₋₄alkyl, —O(C₁₋₄alkyl), —NH(C₁₋₄alkyl), —N(C₁₋₄alkyl)(C₁₋₄alkyl), and —S(O)ₙ(alkyl), wherein said 3- to 7-membered heterocyclic groups contain one or more heteroatom(s) independently selected from N, O, and S, with the point of attachment being either carbon or nitrogen,

R₁ and R₃ are independently selected from hydrogen, halogen, cyano, C₁₋₆alkyl₁, (C₃₋₇cycloalkyl,) C₁₋₄alkyll, —O(C₃₋₇cycloalkyl₁)C₁₋₄alkyl₁,halo(C₁₋₆)alkyl₁, —O(halo(C₁-C₆) alkyl₁), —O(C₁₋₆alkyll), and S(O)ₙ(C₁₋₆alkyl₁), where each said alkyll is straight, branched, or cyclic and may contain 1 or more double or triple bonds, and is optionally substituted by one or more substituents independently chosen from halogen, hydroxy, oxo, cyano, C₁₋₄alkoxy, amino, and mono- or di(C₁₋₄)alkylamino, and where said C₃₋₇cycloalkyl₁ is optionally substituted by one or more substituents independently chosen from halogen, hydroxy, oxo, cyano, C₁₋₄alkoxy, amino, and mono- or di(C₁₋₄)alkylamino

with the proviso that not both R₁ and R₃ are hydrogen; and

Ar is selected from the group consisting of phenyl, naphthyl, pyridyl, pyrimidinyl, and thiophenyl, each of which is mono-, di-, or tri-substituted with R_{C};

R_{A} and R_{B}, which may be the same or different, are independently selected at each occurrence from the group consisting of: hydrogen and straight, branched, or cyclic alkyl groups, including (cycloalkyl)alkyl groups, consisting of 1 to 8 carbon atoms, which may contain one or more double or triple bonds, each of which may be further substituted with one or more substituent(s) independently selected from oxo, hydroxy, halogen, nitro, cyano, C₁₋₆alkoxy, —NH(C₁₋₆alkyl), —N(C₁₋₆alkyl)(C₁₋₆alkyl), —NHC(═O)(C₁₋₆alkyl), —N(C₁₋₆alkyl)C(═O)(C₁₋₆alkyl), —NHS(O)ₙ(C₁₋₆alkyl), —S(O)ₙ(C₁₋₆alkyl), —S(O)ₙNH(C₁₋₆alkyl), —S(O)ₙN(C₁₋₆alkyl)(C₁₋₆alkyl), and Z;

R_{C} is independently selected at each occurrence from halogen, cyano, halo(C₁₋₆)alkyl, halo(C₁₋₆)alkoxy, hydroxy, amino, and C₁₋₆alkyl substituted with 0-2 R_{D}, C₂₋₆alkenyl substituted with 0-2 R_{D}, C₂₋₆alkynyl substituted with 0-2 R_{D}, C₃₋₇cycloalkyl substituted with 0-2 R_{D}, (C₃₋₇cycloalkyl)C₁₋₄alkyl substituted with 0-2 R_{D}, C₁₋₆alkoxy substituted with 0-2 R_{D}, —NH(C₁₋₆alkyl) substituted with 0-2 R_{D}, —N(C₁₋₆alkyl)(C₁₋₆alkyl) each C₁₋₄alkyl independently substituted with 0-2 R_{D}, −XRA, and Y, with the proviso that at least one of the positions ortho or para to the point of attachment of Ar to the pyrimdine ring shown in Formula A is substituted;

R_{D} is independently selected at each occurrence the group consisting of halogen, hydroxy, cyano, C₁₋₄alkyl, —O(C₁₋₄alkyl), —NH(C₁₋₄alkyl), —N(C₁₋₄alkyl)(C₁₋₄alkyl), —S(O)ₙ(alkyl) halo(C₁₋₄) alkyl, halo(C₁₋₄)alkoxy, CO(C₁₋₄alkyl), CONH(C₁₋₄alkyl), CON(C₁₋₄alkyl)(C₁₋₄alkyl), −XRA, and Y;

X is independently selected at each occurrence from the group consisting of —CH₂—, —CHR_{B}—, —O—, —C(═O)—, —C(═O)O—, —S(O)ₙ—, —NH—, —NR_{B}—, —C(═O)NH—, —(═O)NR_{B}—, —S(O)ₙNH—, —S(O)ₙNR_{B}—, —OC(═S)S—, —NHC(═O)—, —NR_{B}C(═O)—, —NHS(O)ₙ—, —OSiHₙ(C₁₋₄alkyl₂₋ₙ)—, and —NR_{B}S(O)ₙ—;

Y and Z are independently selected at each occurrence from the group consisting of: 3- to 7-membered carbocyclic and heterocyclic groups, which are saturated, unsaturated, or aromatic, which may be further substituted with one or more substituents independently selected from halogen, oxo, hydroxy, amino, C₁₋₄alkyl, —O(C₁₋₄alkyl), —NH(C₁₋₄alkyl), —N(C₁₋₄alkyl)(C₁₋₄alkyl), and —S(O)ₙ(alkyl); and n is 0, 1, or 2.

Particular embodiments of the invention include compounds and salts of Formula A in which

R_{X} and R_{Y} are the same or different and are independently selected from:

a) —(C═O)alkyl_{A}, wherein alkyl_{A} is a straight or branched alkyl group having from 1 to 8 carbon atoms;

b) straight, branched, or cyclic alkyl groups, including cycloalkyl(alkyl)groups, said alkyl groups having from 1 to 8 carbon atoms and optionally containing one or more double or triple bonds, each of which alkyl groups may be further substituted with one or more substituent(s) independently selected from:

i) hydroxy, halogen, amino, cyano, —O(C₁₋₄alkyl), —NH(C₁₋₄alkyl), and —NH(C₁₋₄alkyl) (C₁₋₄alkyl), and

ii) 3- to 7-membered carbocyclic and heterocyclic groups, which are saturated, unsaturated, or aromatic, which may be substituted with one or more substituents independently selected from halogen, halo(C₁₋₄)alkyl, halo(C₁₋₄)alkoxy, oxo, hydroxy, amino, C₁₋₄alkyl, —O(C₁₋₄alkyl), —NH(C₁₋₄alkyl), —N(C₁₋₄alkyl) (C₁₋₄alkyl), and —S(O)ₙ(alkyl), wherein said 3- to 7-membered heterocyclic groups contain one or more heteroatom(s) independently selected from N, O, and S, with the point of attachment being either carbon or nitrogen,

R₁ and R₃ are independently selected from C₁₋₆ alkyll, (C₃₋₇cycloalkyl,)C₁₋₄alkyll, —O(C₃₋₇cycloalkyl₁) C₁₋₄alkyl₁, halo(C₁₋₆)alkyl₁, —O(halo(C₁₋₆)alkyl₁), and —O(C₁₋₆alkyll), where each said alkyll is straight, branched, or cyclic and may contain 1 or more double or triple bonds, and is optionally substituted by one or more substituents independently chosen from halogen, hydroxy, oxo, cyano, C₁₋₄alkoxy, amino, and mono- or di(C₁₋₄)alkylamino, and where said C₃₋₇cycloalkyl₁ is optionally substituted by one or more substituents independently chosen from halogen, hydroxy, oxo, cyano, C₁₋₄alkoxy, amino, and mono- or di(C₁₋₄)alkylamino

Ar is phenyl, which is mono-, di-, or tri-substituted with R_{C};

R_{A} and R_{B}, which may be the same or different, are independently selected at each occurrence from the group consisting of: hydrogen and straight, branched, or cyclic alkyl groups, including (cycloalkyl)alkyl groups, consisting of 1 to 8 carbon atoms, which may contain one or more double or triple bonds, each of which may be further substituted with one or more substituent(s) independently selected from oxo, hydroxy, halogen, nitro, cyano, C₁₋₆alkoxy, —NH(C₁₋₆alkyl), —N(C₁₋₆alkyl)(C₁₋₆alkyl), —NHC(═O)(C₁₋₆alkyl), —N(C₁₋₆alkyl)C(═O)(C_{1- 6}alkyl), and Z;

R_{C} is independently selected at each occurrence from halogen, eyano, halo(C₁₋₆)alkyl, halo(C₁₋₆)alkoxy, hydroxy, amino, and C₁₋₆alkyl substituted with 0-2 R_{D}, C₂₋₆ alkenyl substituted with 0-2 R_{D}, C₂₋₆alkynyl substituted with 0-2 R_{D}, C₃₋₇cycloalkyl substituted with 0-2 R_{D}, (C₃₋₇cycloalkyl)C₁₋₄alkyl substituted with 0-2 R_{D}, C₁₋₆alkoxy substituted with 0-2 R_{D}, —NH(C₁₋₆alkyl) substituted with 0-2 R_{D}, —N(C₁₋₆alkyl)(C₁₋₆alkyl) each C₁₋₄alkyl independently substituted with 0-2 R_{D}, −XRA, and Y, with the proviso that at least one of the positions ortho or para to the point of attachment of Ar to the pyrimidine ring shown in Formula A is substituted;

R_{D} is independently selected at each occurrence the group consisting of halogen, hydroxy, cyano, C₁₋₄alkyl, —O(C₁₋₄alkyl), —NH(C₁₋₄alkyl), —N(C₁₋₄alkyl)(C₁₋₄alkyl), halo(C₁₋₄)alkyl, halo(C₁₋₄)alkoxy, CO(C₁₋₄alkyl), CONH(C₁₋₄alkyl), CON(C₁₋₄alkyl)(C₁₋₄alkyl), −XR_{A}, and Y;

X is independently selected at each occurrence from the group consisting of —CH₂—, —CHR_{B}—, —O—, —C(═O)—, —C(═O)O—, —NH—, —NR_{B}—, —C(═O)NH—, —C(═O)NR_{B}—, —NHC(═O)—, and —NR_{B}C(═O)—;

Y and Z are independently selected at each occurrence from the group consisting of: 3- to 7-membered carbocyclic and heterocyclic groups, which are saturated, unsaturated, or aromatic, which may be further substituted with one or more substituents independently selected from halogen, oxo, hydroxy, amino, C₁₋₄alkyl, —O(C₁₋₄alkyl), —NH(C₁₋₄alkyl), and —N(C₁₋₄alkyl)(C₁₋₄alkyl); and n is 0, 1, or 2.

Preferred compounds and salts of Formula A are those in which Ar is phenyl mono-, di-, or tri-substituted with R_{C}, and R₁ and R₃ are independently selected from groups

(1) hydrogen, halogen, C₁₋₄alkoxy, halo(C₁₋₄)alkyl, halo(C₁₋₄)alkoxy, and

(2) C₁₋₆alkyl and (C₃₋₇cycloalkyl)C₁₋₄alkyl, wherein each member of group (2) is unsubstituted or substituted by one to three substituents independently selected from hydroxy, oxo, cyano, C₁₋₄alkoxy, amino, and mono- or di(C₁₋₄)alkylamino.

This class of embodiments of the inventions particularly includes compounds and salts in which R_{X} and R_{Y}, which may be the same or different, are independently selected at each occurrence from straight, branched, or cyclic alkyl groups, including (cycloalkyl)alkyl groups, consisting of 1 to 8 carbon atoms, which may contain one or more double or triple bonds.

Other embodiments of the invention include compounds and salts of Formula A in which Ar is a phenyl group of the formula:

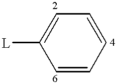

wherein L indicates a bond to the pyrimidine ring of Formula A and the phenyl group is substituted at one, two, or three of positions 2, 4, and 6 positions of the phenyl ring with substituents independently selected from:

i) halogen, cyano, halo(C₁₋₄)alkyl, halo(C₁₋₄)alkoxy, hydroxy, amino, C₁₋₆ alkyl, C₁₋₆alkoxy, (C₁₋₄alkoxy)C₁₋₄alkoxy, and mono- or di(C₁₋₄alkyl)amino,

ii) C₁₋₆alkyl and C₁₋₆alkoxy which are further substituted with a 3- to 7-membered carbocyclic and heterocyclic group, which is saturated, unsaturated, or aromatic, which 3- to 7-membered carbocyclic and heterocyclic group may be further substituted with one or more substituents independently selected from halogen, oxo, hydroxy, amino, C₁₋₄alkyl, —O(C₁₋₄alkyl), —NH(C₁₋₄alkyl), and —N(C₁₋₄alkyl)(C₁₋₄alkyl).

Also provided by the invention are compounds and salts of Formula Ic

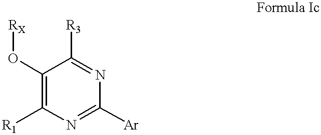

wherein: R_{X} is chosen from straight, branched, or cyclic alkyl groups, including cycloalkyl(alkyl) groups, having from 1 to 8 carbon atoms, which may contain one or more double or triple bonds, each of which may be further substituted with one or more substituent(s) independently selected from:

(a) hydroxy, halogen, amino, cyano, —O(C₁₋₄alkyl), —NH(C₁₋₄alkyl), and —NH(C₁₋₄alkyl) (C₁₋₄alkyl), and

(b) 3- to 7-membered carbocyclic and heterocyclic groups, which are saturated, unsaturated, or aromatic, which may be substituted with one or more substituents selected from halogen, halo(C₁₋₄)alkyl, oxo, hydroxy, amino, C₁₋₄alkyl, —O(C₁₋₄alkyl), —NH(C₁₋₄alkyl), —N(C₁₋₄alkyl) (C₁₋₄alkyl), wherein said 3- to 7-membered heterocyclic groups contain one or more heteroatom(s) selected from N, O, and S, with the point of attachment being either carbon or nitrogen. The remaining variables Ar, R₁ and R₃ are as defined for Formula I, for Formula Ia (preferred) or Formula A. The inventions particularly includes compounds and salts of Formula Ic in which Ar is as defined for Formula Ia and R₁ and R₃ are independently selected from the group consisting of hydrogen, halogen, C₁₋₄alkyl, C₁₋₄alkoxy, and halo(C₁₋₄)alkyl.

Another embodiment of the invention includes compounds and salts of Formula B

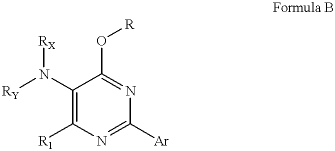

In which Ar is phenyl mono-, di-, or tri-substituted with R_{C} (where R_{C} carries the definition given in Formula Ia); R is selected from straight, branched, or cyclic alkyl groups, including (cycloalkyl)alkyl groups, which may contain 1 or more double or triple bonds, and which are optionally substituted by one or more substituents independently chosen from oxo, hydroxy, halogen, cyano, —O(C₁₋₄alkyl), amino, —NH(C₁₋₄alkyl), and —N(C₁₋₄alkyl)(C₁₋₄alkyl); R₁ is selected from hydrogen, halogen, cyano, C₁₋₄alkyl, (C₃₋₇cycloalkyl)C₁₋₄alkyl, halo(C₁₋₄)alkyl, halo(C₁₋₄)alkoxy, and —O(C₁₋₄alkyl); and R_{X} and R_{Y} are the same or different and are independently selected from:

a) hydrogen,

b) —(C═O)alkyl_{A}, wherein alkyl_{A} is a straight or branched alkyl group having from 1 to 8 carbon atoms;

c) straight, branched, or cyclic alkyl groups, including cycloalkyl(alkyl) groups, having from 1 to 8 carbon atoms, which may contain one or more double or triple bonds, each of which may be further substituted with one or more substituent(s) independently selected from:

i) hydroxy, halogen, amino, cyano, —O(C₁₋₄alkyl), —NH(C₁₋₄alkyl), and —NH(C₁₋₄alkyl)(C₁₋₄alkyl), and

ii) 3- to 7-membered carbocyclic and heterocyclic groups, which are saturated, unsaturated, or aromatic, which may be substituted with one or more substituents selected from halogen, halo(C₁₋₄)alkyl, halo(C₁₋₄)alkoxy, oxo, hydroxy, amino, C₁₋₄alkyl, —O(C₁₋₄alkyl), —NH(C₁₋₄alkyl), —N(C₁₋₄alkyl)(C₁₋₄alkyl), and —S(O)ₙ(alkyl), wherein said 3- to 7-membered heterocyclic groups contain one or more heteroatom(s) independently selected from N, O, and S, with the point of attachment being either carbon or nitrogen.

Preferred compound of Formula B are those in which Ar is a phenyl group of the formula:

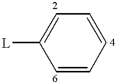

wherein L indicates a bond to the pyrimidine ring in Formula B and the Ar phenyl group is substituted at one, two, or three of positions 2, 4, and 6 with substituents independently selected from (i) halogen, cyano, halo(C₁₋₄)alkyl, halo(C₁₋₄)alkoxy, hydroxy, amino, C₁₋₆alkyl, C₁₋₆alkoxy, (C₁₋₄alkoxy)C₁₋₄alkoxy, and mono- or di(C₁₋₄alkyl) amino, and (ii) C₁₋₆alkyl and C₁₋₆alkoxy which are further substituted with a 3- to 7-membered carbocyclic and heterocyclic group, which is saturated, unsaturated, or aromatic, which 3- to 7-membered carbocyclic and heterocyclic group may be further substituted with one or more substituents independently selected from halogen, oxo, hydroxy, amino, C₁₋₄alkyl, —O(C₁₋₄alkyl), —NH(C₁₋₄alkyl), and —N(C₁₋₄alkyl)(C₁₋₄alkyl).

Preferred compound and salts of this embodiment of the invention include compounds in which R_{X} and R_{Y} are the same or different and are independently selected from the groups (a) hydrogen (with the proviso that R_{X} and R_{Y} are not both hydrogen), (b) —(C═O)alkyl_{A}, wherein alkyl_{A} is a straight or branched alkyl group having from 1 to 8 carbon atoms; and (c) straight, branched, or cyclic alkyl groups, including cycloalkyl(alkyl), said straight, branched, or cyclic alkyl groups having from 1 to 8 carbon atoms, and containing zero, one or more double or triple bonds, each of which 1 to 8 carbon atoms may be further substituted with one or more substituent(s) independently selected from hydroxy, halogen, amino, cyano, —O(C₁₋₄alkyl), —NH(C₁₋₄alkyl), and —NH(C₁₋₄alkyl)(C₁₋₄alkyl).

Additional preferred compounds of Formula B are those wherein Ar is a phenyl group of the formula:

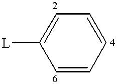

wherein L indicates a bond to the pyrimidine ring in Formula B and the Ar phenyl group is substituted at one, two, or three of positions 2, 4, and 6 with substituents independently selected from:

i) halogen, cyano, halo(C₁₋₄)alkyl, halo(C₁₋₄)alkoxy, hydroxy, amino, C₁₋₆alkyl, C₁₋₆alkoxy, (C₁₋₄alkoxy)C₁₋₄alkoxy, and mono- or di(C₁₋₄alkyl)amino,

ii) C₁₋₆ alkyl and C₁₋₆alkoxy which are further substituted with a 3- to 7-membered carbocyclic and heterocyclic group, which is saturated, unsaturated, or aromatic, which 3- to 7-membered carbocyclic and heterocyclic group may be further substituted with one or more substituents independently selected from halogen, oxo, hydroxy, amino, C₁₋₄alkyl, —O(C₁₋₄alkyl), —NH(C₁₋₄alkyl), and —N(C₁₋₄alkyl)(C₁₋₄alkyl);

R_{X} and R_{Y} are the same or different and are independently selected from the group consisting of:

a) hydrogen (with the proviso that R_{X} and R_{Y} are not both hydrogen),

b) —(C═O)alkyl_{A}, wherein alkyl_{A} is a straight or branched alkyl group having from 1 to 8 carbon atoms;

c) straight, branched, or cyclic alkyl groups, including cycloalkyl(alkyl) groups, said straight, branched, or cyclic alkyl groups have from 1 to 8 carbon atoms and may contain one or more double or triple bonds.

The invention further provides compounds and salts of Formula C

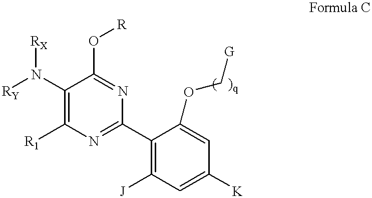

wherein R, R₁, R_{X}, and R_{Y} carry the definitions set forth for Formula B and q is an integer from 1 to 4; G is hydrogen, hydroxy, C₁₋₆alkoxy, —NH(C₁₋₆alkyl), —N(C₁₋₆alkyl)(C₁₋₆alkyl), or a 3- to 7-membered carbocyclic or heterocyclic group which is saturated, unsaturated, or aromatic, which is unsubstituted or substituted with one or more substituents independently selected from halogen, halo(C₁₋₄)alkyl, halo(C₁₋₄)alkoxy, oxo, hydroxy, amino, C₁₋₄alkyl, —O(C₁₋₄alkyl), —NH(C₁₋₄alkyl), —N(C₁₋₄alkyl)(C₁₋₄alkyl), and —S(O)ₙ(alkyl), wherein said 3- to 7-membered heterocyclic group contains one or more heteroatom(s) independently selected from N, O, and S, with the point of attachment being either carbon or nitrogen;

J and K are independently selected from halogen, cyano, halo(C₁₋₄)alkyl, halo(C₁₋₄)alkoxy, hydroxy, amino, C₁₋₆alkyl, C₁₋₄alkyl, C₁₋₄alkoxy, (C₁₋₄alkoxy) C₁₋₄alkoxy, and mono- or di(C₁₋₄alkyl)amino.

In yet another embodiment the invention provides compounds and salts of Formula D

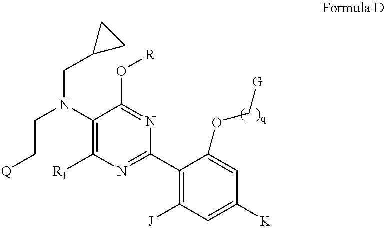

wherein R and R₁ carry the definitions set forth for Formula B: Q is hydrogen, C₃₋₇cycloalkyl, pyrrolidinyl, piperidinyl, morpholino, or piperazinyl; q is an integer from 1 to 4; G is hydrogen, hydroxy, C₁₋₆alkoxy, —NH(C₁₋₆alkyl), —N(C₁₋₆alkyl)(C₁₋₆alkyl), or a 3- to 7-membered carbocyclic or heterocyclic group, which is saturated, unsaturated, or aromatic, which is unsubstituted or substituted with one or more substituents independently selected from halogen, halo(C₁₋₄)alkyl, halo(C₁₋₄)alkoxy, oxo, hydroxy, amino, C₁₋₄alkyl, —O(C₁₋₄alkyl), —NH(C₁₋₄alkyl), —N(C₁₋₄alkyl)(C₁₋₄alkyl), and —S(O)ₙ(alkyl), wherein said 3- to 7-membered heterocyclic group contains one or more heteroatom(s) independently selected from N, O, and S, with the point of attachment being either carbon or nitrogen;

J and K are independently selected from halogen, cyano, halo(C₁₋₄)alkyl, halo(C₁₋₄)alkoxy, hydroxy, amino, C₁₋₆alkyl, C₁₋₄alkyl, C₁₋₄alkoxy, (C₁₋₄alkoxy) C₁₋₄alkoxy, and mono- or di(C₁₋₄alkyl)amino; and

R_{X} and R_{Y} are the same or different and are independently selected from hydrogen (with the proviso that R_{X} and R_{Y} are not both hydrogen) and straight, branched, or cyclic alkyl groups having from 1 to 6 carbon atoms, which alkyl groups may contain one or more double or triple bonds.

Other compound and salts of the invention are those having Formula E

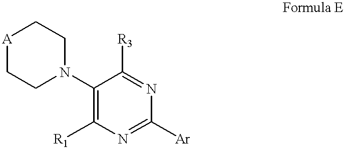

wherein R₁, R₃, and Ar may carry the definitions set forth for Formula Ia and A is NH, N(C₁₋₆-alkyl), CH₂, CH(C₁₋₆-alkyl) or O.

Compounds of the invention are useful in treating a variety of conditions including affective disorders, anxiety disorders, stress disorders, eating disorders, and drug addiction.

Affective disorders include all types of depression, bipolar disorder, cyclothymia, and dysthymia.

Anxiety disorders include generalized anxiety disorder, panic, phobias and obsessive-compulsive disorder.

Stress-related disorders include post-traumatic stress disorder, hemorrhagic stress, stress-induced psychotic episodes, psychosocial dwarfism, stress headaches, stress-induced immune systems disorders such as stress-induced fever, and stress-related sleep disorders.

Eating disorders include anorexia nervosa, bulimia nervosa, and obesity.

Modulators of the CRF receptors may also be useful in the treatment of a variety of neurological disorders including supranuclear palsy, AIDS related dementias, multiinfarct dementia, neurodegenerative disorders such as Alzheimers disease, Parkinsons disease, and Huntingtons disease, head trauma, spinal cord trauma, ischemic neuronal damage, amyotrophic lateral sclerosis, disorders of pain perception such as fibromyalgia and epilepsy.

Additionally compounds of Formula I are useful as modulators of the CRF receptor in the treatment of a number of gastrointestinal, cardiovascular, hormonal, autoimmune and inflammatory conditions. Such conditions include irritable bowel syndrome, ulcers, Crohns disease, spastic colon, diarrhea, post operative ilius and colonic hypersensitivity associated with psychopathological disturbances or stress, hypertension, tachycardia, congestive heart failure, infertility, euthyroid sick syndrome, inflammatory conditions effected by rheumatoid arthritis and osteoarthritis, pain, asthma, psoriasis and allergies.

Compounds of Formula I are also useful as modulators of the CRF, receptor in the treatment of animal disorders associated with aberrant CRF levels. These conditions include porcine stress syndrome, bovine shipping fever, equine paroxysmal fibrillation, and dysfunctions induced by confinement in chickens, sheering stress in sheep or human-animal interaction related stress in dogs, psychosocial dwarfism and hypoglycemia.

Typical subjects to which compounds of the invention may be administered will be mammals, particularly primates, especially humans. For veterinary applications, a wide variety of subjects will be suitable, e.g. livestock such as cattle, sheep, goats, cows, swine and the like; poultry such as chickens, ducks, geese, turkeys, and the like; and domesticated animals particularly pets such as dogs and cats. For diagnostic or research applications, a wide variety of mammals will be suitable subjects including rodents (e.g. mice, rats, hamsters), rabbits, primates, and swine such as inbred pigs and the like. Additionally, for in vitro applications, such as in vitro diagnostic and research applications, body fluids and cell samples of the above subjects will be suitable for use such as mammalian, particularly primate such as human, blood, urine or tissue samples, or blood urine or tissue samples of the animals mentioned for veterinary applications.

The CRF binding compounds provided by this invention and labeled derivatives thereof are also useful as standards and reagents in determining the ability of a potential pharmaceutical to bind to the CRF receptor.

Labeled derivatives the CRF antagonist compounds provided by this invention are also useful as radiotracers for positron emission tomography (PET) imaging or for single photon emission computerized tomography (SPECT).

The present invention also pertains to methods of inhibiting the binding of CRF to CRF receptors which methods involve contacting a solution containing compound of the invention with cells expressing CRF receptors, wherein the compound is present in the solution at a concentration sufficient to inhibit CRF binding to CRF receptors in vitro. This method includes inhibiting the binding of CRF to CRF receptors in vivo, e.g., in a patient given an amount of a compound of Formula I that would be sufficient to inhibit the binding of CRF to CRF receptors in vitro. In one embodiment, such methods are useful in treating physiological disorders associated with excess concentrations of CRF. The amount of a compound that would be sufficient to inhibit the binding of a CRF to the CRF receptor may be readily determined via a CRF receptor binding assay (see Example 96), or from the EC₅₀ of a CRF receptor functional assay, such as a standard assay of CRF receptor mediated chemotaxis. The CRF receptors used to determine in vitro binding may be obtained from a variety of sources, for example from cells that naturally express CRF receptors, e.g. IMR32 cells or from cells expressing cloned human CRF receptors.

The present invention also pertains to methods for altering the activity of CRF receptors, said method comprising exposing cells expressing such receptors to an effective amount of a compound of the invention, wherein the compound is present in the solution at a concentration as sufficient to specifically alter the signal transduction activity in response to CRF in cells expressing high levels of CRF1 receptors in vitro. This method includes altering the signal transduction activity of CRF receptors in vivo, e.g., in a patient given an amount of a compound of Formula I that would be sufficient to alter the signal transduction activity in response to CRF in cells expressing high levels of CRF1 in vitro. The amount of a compound that would be sufficient to alter the signal transduction activity in response to CRF receptors may be determined via an assay of CRF receptor mediated signal transduction, such as an assay wherein the binding of CRF to a cell surface CRF receptor effects a changes in reporter gene expression.

The present invention also pertains to packaged pharmaceutical compositions for treating disorders responsive to C5a receptor modulation, e.g., eating disorders, depression or stress. The packaged pharmaceutical compositions include a container holding a therapeutically effective amount of at least one CRF1 receptor modulator as described supra and instructions for using the treating disorder responsive to CRF1 receptor modulation in the patient.

Definitions

The compounds herein described may have one or more asymmetric centers or planes. Compounds of the present invention containing an asymmetrically substituted atom may be isolated in optically active or racemic forms. It is well known in the art how to prepare optically active forms, such as by resolution of racemic forms (racemates), by asymmetric synthesis, or by synthesis from optically active starting materials. Resolution of the racemates can be accomplished, for example, by conventional methods such as crystallization in the presence of a resolving agent, or chromatography, using, for example a chiral HPLC column. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral (enantiomeric and diastereomeric), and racemic forms, as well as all geometric isomeric forms of a structure are intended, unless the specific stereochemistry or isomeric form is specifically indicated.

When any variable occurs more than one time in any constituent or formula for a compound, its definition at each occurrence is independent of its definition at every other occurrence. Thus, for example, if a group is shown to be substituted with 0-2 R^{*}, then said group may optionally be substituted with up to two R^{*} groups and R^{*} at each occurrence is selected independently from the definition of R^{*}. Also, combinations of substituents and/or variables are permissible only if such combinations result in stable compounds.

Compounds of Formula I include, but are not limited to, compounds of Formula Ia, Ic, A, B, C, D, and E.

As indicated above, various substituents of the various formulae are optionally substituted, including Ar, R₁, R₂, and R₃ of Formula I and subformulae thereof, and such substituents as recited in the sub-formulae such as Formula I and subformulae, e.g. Formula Ia, Ic, A, B, C, D, and E and the like. The term substituted, as used herein, means that any one or more hydrogens on the designated atom is replaced with a selection from the indicated group, provided that the designated atoms normal valence is not exceeded, and that the substitution results in a stable compound. When a substituent is keto (i.e., =O), then 2 hydrogens on the atom are replaced. Keto substituents are not present on aromatic moieties. The present invention is intended to include all isotopes of atoms occurring in the present compounds. Isotopes include those atoms having the same atomic number but different mass numbers, By way of general example and without limitation, isotopes of hydrogen include tritium and deuterium. Isotopes of carbon include ¹¹C, ¹³C, and ¹⁴C.

When substituted, those substituents (Ar, R₁, R₂, and R₃) may be substituted by other than hydrogen at one or more available positions, typically 1 to 3 or 4 positions, by one or more suitable groups such as those disclosed herein. Suitable groups that may be present on a substituted Ar, R₁, R₂, and R₃ group or other substituent include e.g. halogen such as fluoro, chloro, bromo and iodo; cyano; hydroxyl; nitro; azido; alkanoyl such as a C₁₋₆alkanoyl group such as acyl and the like; carboxamido; alkyl groups, including cycloalkyl groups, having 1 to about 12 carbon atoms, or 1, 2, 3, 4, 5, or 6 carbon atoms; alkenyl and alkynyl groups including groups having one or more unsaturated linkages and from 2 to about 12 carbon, or 2, 3, 4, 5 or 6 carbon atoms; alkoxy groups having those having one or more oxygen linkages and from I to about 12 carbon atoms, or 1, 2, 3, 4, 5 or 6 carbon atoms; aryloxy such as phenoxy; alkylthio groups including those moieties having one or more thioether linkages and from 1 to about 12 carbon atoms, or 1, 2, 3, 4, 5 or 6 carbon atoms; alkylsulfinyl groups including those moieties having one or more sulfinyl linkages and from 1 to about 12 carbon atoms, or 1, 2, 3, 4, 5, or 6 carbon atoms; alkylsulfonyl groups including those moieties having one or more sulfonyl linkages and from 1 to about 12 carbon atoms, or 1, 2, 3, 4, 5, or 6 carbon atoms; aminoalkyl groups such as groups having one or more N atoms and from 1 to about 12 carbon atoms, or 1, 2, 3, 4, 5 or 6 carbon atoms; carbocyclic aryl having 6 or more carbons, particularly phenyl (e.g. an Ar group being a substituted or unsubstituted biphenyl moiety); aralkyl having 1 to 3 separate or fused rings and from 6 to about 18 carbon ring atoms, with benzyl being a preferred group; aralkoxy having 1 to 3 separate or fused rings and from 6 to about 18 carbon ring atoms, with 0-benzyl being a preferred group; or a saturated, unsaturated, or aromatic heterocyclic group having 1 to 3 separate or fused rings with 3 to about 8 members per ring and one or more N, O or S atoms, e.g. coumarinyl, quinolinyl, pyridyl, pyrazinyl, pyrimidyl, furyl, pyrrolyl, thienyl, thiazolyl, oxazolyl, imidazolyl, indolyl, benzofuranyl, benzothiazolyl, tetrahydrofuranyl, tetrahydropyranyl, piperidinyl, morpholino and pyrrolidinyl. Such heterocyclic groups may be further substituted, e.g. with hydroxy, alkyl, halogen and amino.

As used herein, alkyl is intended to include both branched and straight-chain saturated aliphatic hydrocarbon groups, having the specified number of carbon atoms. Examples of alkyl include, but are not limited to, methyl, ethyl, n-propyl, i-propyl, n-butyl, s-butyl, t-butyl, n-pentyl, and s-pentyl. Preferred alkyl groups are C₁-C₆alkyl groups. Especially preferred alkyl groups are methyl, ethyl, propyl, butyl, 3-pentyl. The term C₁₋₄alkyl as used herein includes alkyl groups consisting of 1 to 4 carbon atoms, which may contain a cyclopropyl moiety. Suitable examples are methyl, ethyl, and cyclopropylmethyl.

“Cycloalkyl” is intended to include saturated ring groups, having the specified number of carbon atoms, such as cyclopropyl, cyclobutyl, cyclopentyl, or cyclohexyl. Cycloalkyl groups typically will have 3 to about 8 ring members.

In the term (C₃₋₆cycloalkyl)C₁₋₄alkyl, as defined above, the point of attachment is on the alkyl group. This term encompasses, but is not limited to, cyclopropylmethyl, cyclohexylmethyl, cyclohexylmethyl.

“Alkenyl” is intended to include hydrocarbon chains of either a straight or branched configuration comprising one or more unsaturated carbon-carbon bonds which may occur in any stable point along the chain, such as ethenyl and propenyl. Alkenyl groups typically will have 2 to about 12 carbon atoms, more typically 2 to about 8 carbon atoms.

“Alkynyl” is intended to include hydrocarbon chains of either a straight or branched configuration comprising one or more triple carbon-carbon bonds which may occur in any stable point along the chain, such as ethynyl and propynyl. Alkynyl groups typically will have 2 to about 12 carbon atoms, more typically 2 to about 8 carbon atoms.

“Haloalkyl” is intended to include both branched and straight-chain saturated aliphatic hydrocarbon groups having the specified number of carbon atoms, substituted with 1 or more halogen (for example −CᵥF_{w} where v=1 to 3 and w=1 to (2v+1). Examples of haloalkyl include, but are not limited to, trifluoromethyl, trichloromethyl, pentafluoroethyl, and pentachloroethyl. Typical haloalkyl groups will have 1 to about 8 carbon atoms, more typically 1 to about 6 carbon atoms.

“Alkoxy” represents an alkyl group as defined above with the indicated number of carbon atoms attached through an oxygen bridge. Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Alkoxy groups typically have 1 to about 12 carbon atoms, more typically 1 to about 8 carbon atoms.

As used herein, the term alkylthio includes those groups having one or more thioether linkages and suitably from 1 to about 12 carbon atoms, more typically 1 to about 8 carbon atoms, still more typically 1 to about 6 carbon atoms.

As used herein, the term “alkylsulfinyl” includes those groups having one or more sulfoxide (SO) linkage groups and suitably from 1 to about 12 carbon atoms, more typically 1 to about 8 carbon atoms, still more typically 1 to about 6 carbon atoms.

As used herein, the term “alkylsulfonyl” includes those groups having one or more sulfonyl (SO₂) linkage groups and suitably from 1 to about 16 carbon atoms, more typically 1 to about 12 carbon atoms, still more typically 1 to about 6 or 8 carbon atoms.

As used herein, the term “alkylamino” includes those groups having one or more primary, secondary and/or tertiary amine groups and suitably from 1 to about 12 carbon atoms, more typically 1 to about 8 carbon atoms, still more typically I to about 6 carbon atoms.

“Halo” or “halogen” as used herein refers to fluoro, chloro, bromo, and iodo; and “counter-ion” is used to represent a small, negatively charged species such as chloride, bromide, hydroxide, acetate, sulfate, and the like.

As used herein, carbocyclic group is intended to mean any stable 3- to 7-membered monocyclic or bicyclic or 7-to 13-membered bicyclic or tricyclic, any of which may be saturated, partially unsaturated, or aromatic. Examples of such carbocycles include, but are not limited to, cyclopropyl, cyclobutyl, cyclopentyl, cyclohexyl, cycloheptyl, adamantyl, cyclooctyl, [3.3.0]bicyclooctane, [4.3.0]bicyclononane, [4.4.0]bicyclodecane, [2.2.2]bicyclooctane, fluorenyl, phenyl, naphthyl, indanyl, adamantyl, and tetrahydronaphthyl.

As used herein, the term heterocyclic group is intended to mean a stable 5-to 7-membered monocyclic or bicyclic or 7-to 10-membered bicyclic heterocyclic ring which is saturated, partially unsaturated, or unsaturated (aromatic), and which consists of carbon atoms and from 1 to 4 heteroatoms independently selected from the group consisting of N, O and S and including any bicyclic group in which any of the above-defined heterocyclic rings is fused to a benzene ring. The nitrogen and sulfur heteroatoms may optionally be oxidized. The term or “heterocycloalkyl” is used to refer to saturated heterocyclic groups.

The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quatemized. It is preferred that when the total number of S and O atoms in the heterocycle exceeds 1, then these heteroatoms are not adjacent to one another. It is preferred that the total number of S and O atoms in the heterocycle is not more than 1. As used herein, the term “aromatic heterocyclic system” is intended to mean a stable 5-to 7-membered monocyclic or bicyclic or 7-to 10-membered bicyclic heterocyclic aromatic ring which consists of carbon atoms and from 1 to 4 heteroatoms independently selected from the group consisting of N, O and S. It is preferred that the total number of S and O atoms in the aromatic heterocycle is not more than 1.

Examples of heterocycles include, but are not limited to, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H, 6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl;- 1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H- 1 ,2,5-thiadiazinyl, 1,2,3 -thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

Preferred heterocyclic groups include, but are not limited to, pyridinyl, pyrimidinyl, furanyl, thienyl, pyrrolyl, pyrazolyl, pyrrolidinyl, morpholinyl, piperidinyl, piperazinyl, and imidazolyl. Also included are fused ring and spiro compounds containing, for example, the above heterocycles.

As used herein, the term carbocyclic aryl includes groups that contain 1 to 3 separate or fused rings and from 6 to about 18 ring atoms, without hetero atoms as ring members. Specifically preferred carbocyclic aryl groups include phenyl, and naphthyl including 1-napthyl and 2-naphthyl.

The phrase “pharmaceutically acceptable” is employed herein to refer to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio. As used herein, pharmaceutically acceptable salts refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, malefic, hydroxymaleic, phenylacetic, glutamic, benzoic, salicylic, sulfanilic, 2-acetoxybenzoic, flmaric, toluenesulfonic, methanesulfonic, ethane disulfonic, oxalic, isethionic, HOOC—(CH₂)n-COOH where n is 0-4, and the like. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remingtons Pharmaceutical Sciences, 17th ed., Mack Publishing Company, Easton, Pa., p. 1418 (1985).

“Prodrugs” are intended to include any covalently bonded carriers which release the active parent drug according to Formula I in vivo when such prodrug is administered to a mammalian subject. Prodrugs of a compound of formula I are prepared by modifying functional groups present in the compound in such a way that the modifications are cleaved, either in routine manipulation or in vivo, to the parent compound. Prodrugs include compounds of formula I wherein a hydroxy, amino, or sulfhydryl group is bonded to any group that, when the prodrug or compound of formula I is administered to a mammalian subject, cleaves to form a free hydroxyl, free amino, or free sulfhydryl group, respectively. Examples of prodrugs include, but are not limited to, acetate, formate and benzoate derivatives of alcohol and amine functional groups in the compounds of formula I, and the like.

Combinations of substituents and/or variables are permissible only if such combinations result in stable compounds. A stable compound or stable structure is meant to imply a compound that is sufficiently robust to survive isolation to a useful degree of purity from a reaction mixture, and formulation into an effective therapeutic agent. The term therapeutically effective amount of a compound of this invention means an amount effective to antagonize abnormal level of CRF or treat the symptoms of affective disorder, anxiety or depression in a host.

Pharmaceutical Preparations

The compounds of general Formula I may be administered orally, topically, parenterally, by inhalation or spray or rectally in dosage unit formulations containing conventional non-toxic pharmaceutically acceptable carriers, adjuvants and vehicles. The term parenteral as used herein includes subcutaneous injections, intravenous, intramuscular, intrathecal injection or infusion techniques. In addition, there is provided a pharmaceutical formulation comprising a compound of general Formula I and a pharmaceutically acceptable carrier. One or more compounds of general Formula I may be present in association with one or more non-toxic pharmaceutically acceptable carriers and/or diluents and/or adjuvants and if desired other active ingredients. The pharmaceutical compositions containing compounds of general Formula I may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropylmethylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be sterile injectable solution or suspension in a non-toxic parentally acceptable dilutent or solvent, for example as a solution in 1,3-butanediol. Among the acceptable vehicles and solvents that may be employed are water, Ringers solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of general Formula I may also be administered in the form of suppositories for rectal administration of the drug. These compositions can be prepared by mixing the drug with a suitable non-irritating excipient that is solid at ordinary temperatures but liquid at the rectal temperature and will therefore melt in the rectum to release the drug. Such materials are cocoa butter and polyethylene glycols.

Compounds of general Formula I may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient.

Frequency of dosage may also vary depending on the compound used and the particular disease treated. However, for treatment of most CNS disorders, a dosage regimen of 4 times daily or less is preferred. For the treatment of stress and depression a dosage regimen of 1 or 2 times daily is particularly preferred.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

Preferred compounds of the invention will have certain pharmacological properties. Such properties include, but are not limited to oral bioavailability, low toxicity, low serum protein binding and desirable in vitro and in vivo half-lifes. Penetration of the blood brain barrier for compounds used to treat CNS disorders is necessary, while low brain levels of compounds used to treat periphereal disorders are often preferred.

Assays may be used to predict these desirable pharmacological properties. Assays used to predict bioavailability include transport across human intestinal cell monolayers, including Caco-2 cell monolayers. Toxicity to cultured hepatocyctes may be used to predict compound toxicity. Penetration of the blood brain barrier of a compound in humans may be predicted from the brain levels of the compound in laboratory animals given the compound intravenously.

Serum protein binding may be predicted from albumin binding assays. Such assays are described in a review by Oravcova, et al. (Journal of Chromatography B (1996) volume 677, pages 1-27).

Compound half-life is inversely proportional to the frequency of dosage of a compound. In vitro half-lives of compounds may be predicted from assays of microsomal half-life as described by Kuhnz and Gieschen (Drug Metabolism and Disposition, (1998) volume 26, pages 1120-1127).

As discussed above, preferred arylpyrimidines of the invention exhibit good activity in standard in vitro CRF receptor binding assays, specifically the assay as specified in Example 96, which follows. References herein to standard in vitro receptor binding assay are intended to refer to that protocol as defined in Example 96 which follows. Generally preferred compounds preferred arylpyrimidines of the invention have an IC₅₀ of about 1 micromolar or less, still more preferably and IC₅₀ of about 100 nanomolar or less even more preferably an IC₅₀ of about 10 nanomolar or less or even 1 nanomolar or less in such a defined standard in vitro CRF receptor binding assay as exemplified by Example 96 which follows.

### EXAMPLES

Preparation of Arylpyrimidines

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. Preferred methods include but are not limited to those methods described below. Each of the references cited below are hereby incorporated herein by reference. Preferred methods for the preparation of compounds of the present invention include, but are not limited to, those described in Scheme I to Scheme IV. Those who are skilled in the art will recognize that the starting materials may be varied and additional steps employed to produce compounds encompassed by the present invention. All references cited herein are hereby incorporated in their entirety herein by reference. The following abbreviations are used herein:

| | |
|---|---|
| AcOH | acetic acid |
| DMF | N,N-dimethylformamide |
| Et₂O | diethyl ether |
| EtOAc | ethyl acetate |
| EtOH | ethanol |
| NaH | sodium hydride |
| NaHMDS | sodium bis(trimethylsilyl)amide |
| THF | tetrahydrofuran |
| EX# | example number |

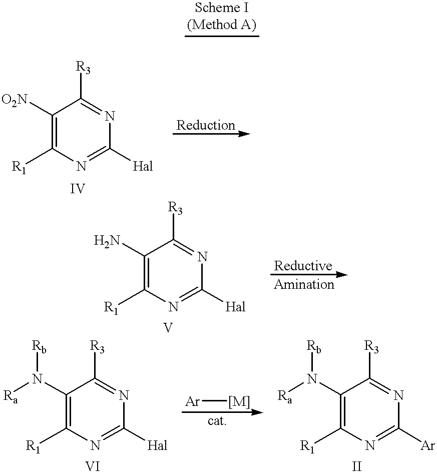

According to the general method A, wherein R₁ and R₃ are as defined for formula I and Hal represents a halogen atom, suitably chloride or bromide. Compounds of formula IV can be prepared according to a known literature procedure (Ref: Journal of Organic Chemistry 1983, 48, 1060). Reduction of the nitro group in IV may be accomplished by a variety of methods known in the art, including hydrogenation with hydrogen and transition metal catalysts or the use of sodium hydrosulfite in aqueous solutions to give V. The amino pyrmidine V may be transformed into VI by reductive amination using aldehydes and reducing agents such as sodium triacetoxyborohydride in inert solvents. The halopyfimidine VI can be converted to arylpyrimidine II by a transition metal-catalyzed coupling reaction with a metalloaryl reagent (Ar-[M]). More commonly employed reagent/catalyst pairs include aryl boronic acid/palladium(0) (Suzuki reaction; N. Miyaura and A. Suzuki, Chemical Review 1995, 95 2457), aryl trialkylstannane/palladium(0) (Stille reaction; T. N. Mitchell, Synthesis 1992, 803), arylzinc/palladium(0) and aryl Grignard/nickel(II). Palladium(0) represents a catalytic system made of a various combination of metal/ligand pair which includes, but not limited to, tetrakis(triphenylphosphine)palladium(0), palladium(II) acetate/tri(o-tolyl)phosphine, tris(dibenzylideneacetone)dipalladium(0)/tri-tert-butylphosphine and dichloro[1,1′-bis(diphenylphosphine) ferrocene]palladium(0). Nickel(II) represents a nickel-containing catalyst such as [1,2-bis(diphenylphosphino)ethane]dichloronickel(II) and [1,3-bis(diphenylphosphino) propane]dichloronickel(II).

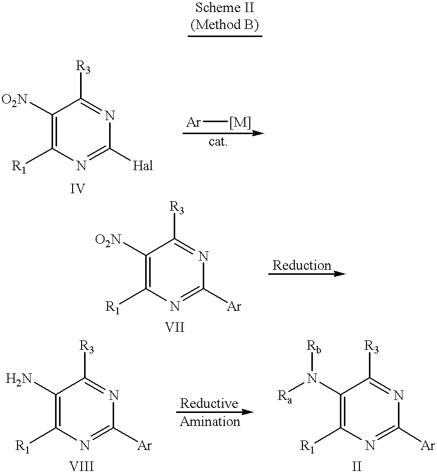

By changing the sequence of events in Scheme I, but using same the methods described in Method A, compounds of the formula II can also be prepared as outlined in Scheme II

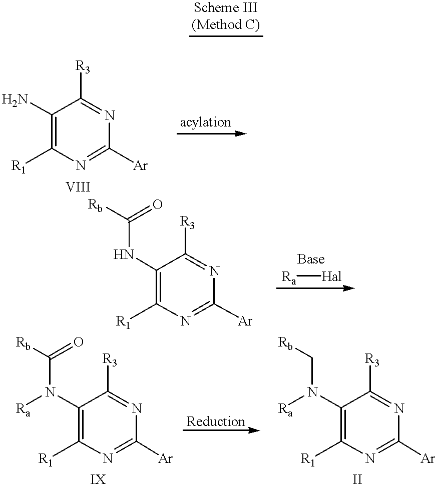

An alternative method for introducing the substituents R_{A} and R_{B}to give compounds of the formula II is outlined in Scheme III and may be accomplished by a variety of methods known in the art. These include reaction of the amine VIII with acid chlorides or anhydrides in the presence of bases such as but not limited to triethylamine or pyridine in inert solvents such as dichloromethane or toluene. The N-H group is then deprotonated by a strong base such as but not limited to alkali metal hydride, alkali metal amide, or alkali metal alkoxide in inert solvents such as but not limited to THF, DMF, or methyl sulfoxide.

Alkylation may be conducted using alkyl halide, suitably bromide or iodide, at temperatures ranging from 0° C. to 100° C. Reduction of the amide IX with reducing agents such as but not limited to lithium aluminum hydride, borane or diisobutylaluminum hydride in inert solvents such as but not limited to THF, ether, or toluene furnishes compounds of the formula II.

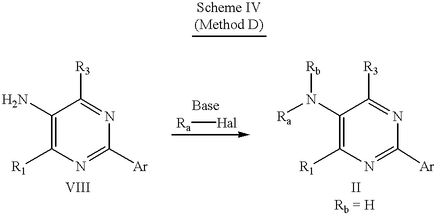

Yet another method for preparing compounds of formula II is illustrated in the Scheme IV. Treatment of the amine VIII with base such as but not limited to alkali metal hydride, alkali metal amide, alkali metal alkoxide or alkali metal carbonates in inert solvents such as but not limited to THF, DMF, methyl sulfoxide or acetonitrile with or without the addition of alkali metal iodide, followed by alkylation with alkyl halide, suitably bromide or iodide, or sulfonate at temperatures ranging from 0° C. to 100° C. furnishes compounds of the formula II.

### EXAMPLES

The preparation of the compounds of the present invention is illustrated further by the following examples, which are not to be construed as limiting the invention in scope or spirit to the specific procedures and compounds described in them.

Commercial reagents are used without further purification, room or ambient temperature refers to 20 to 25° C. Concentration in vacuo implies the use of a rotary evaporator. TLC refers to thin layer chromatography. Proton nuclear magnetic resonance (¹H NMR) spectral data are obtained at 300 or 400 MHz. Mass spectral data are obtained either by CI or APCI methods.

### Example 1

[2-(2,4-dimethoxyphenyl)-4-methoxy-6-methylpyrimidin-5-yl]dipropylamine [Formula I: Ar=2,4-dimethoxyphenyl; R₁=OCH₃; R₂=N(CH₂CH₂CH₃)₂; R₃=CH₃]

A. To a solution of 2-chloro-4-methoxy-6-methyl-5-nitropyrimidine (1.01 g, 5.00 mmol) in THF (20 mL) and water (20 mL) is added sodium hydrosulfite (8.6 g, 50.0 mmol). The mixture is stirred at room temperature for 2.5 h, diluted with saturated aqueous sodium bicarbonate and extracted with ethyl acetate. Combined extracts are washed with brine, dried (sodium sulfate), filtered and concentrated to give 2-chloro-4-methoxypyrimidine-5-ylamine (600 mg): ¹H NMR (CDCl₃, 400 MHz) δ2.38 (s, 3H), 4.02 (s, 3H).

B. To a solution of 2-chloro-4-methoxypyrimidine-5-ylamine (400 mg, 2.3 mmol) in 1,2-dichloroethane (60 mL) is added propionaldehyde (700 mg, 12 mmol) and glacial acetic acid (660 mg). After 10 minutes sodium triacetoxyborohydride (2.5g, 12 mmol) is added in one portion. After 3 h the volatiles are removed by rotary evaporation. The residue is partitioned between ethyl acetate and saturated aqueous sodium bicarbonate, the layers are separated and the aqueous layer further extracted with ethyl acetate. The combined organics are washed with water, brine, dried (sodium sulfate), filtered and concentrated to give (2-chloro-4-methoxy-6-methylpyrimidine-5-yl) dipropylamine (566 mg): ¹H NMR (CDCl₃, 400 MHz) δ0.85 (t, 6H), 1.3 (m, 4H), 2.4 (s, 3H), 2.82 (t, 4H), 4.0 (s, 3H); MS (CI) 258.

C. A stirred solution of (2-chloro-4-methoxy-6-methylpyrimidine-5-yl)dipropylamine (380 mg; 1.47 mmol) and tetrakis(triphenylphosphine)palladium(0) (33 mg; 2 mol %) in ethyleneglycol dimethyl ether (8 mL) is stirred at room temperature for 15 min, then 2,4-dimethoxybenzeneboronic acid (1.76 mmol) and an aqueous solution of sodium carbonate (1.0 M, 4 mL) are added sequentially. The mixture is heated to 75° C. with stirring for 1.5 h, then diluted with 0.1 N sodium hydroxide and extracted twice with 1:1 hexane-ethyl ether. Combined extracts are dried (sodium sulfate), filtered, concentrated, and chromatographed on silica (1:1 hexane-ether) to give the title compound (0.50 g): ¹H NMR (CDCl₃, 400 MHz) δ0.85 (t, 6H), 1.4 (m, 4H), 2.58 (s, 3H), 2.9 (t, 4H), 3.82 (s, 3H), 3.84 (s, 3H), 4.0 (s, 3H), 6.58 (m, 2H), 7.8 (d, 1H); MS (CI) 360.

The EX#s 2-6b in the Table I may be prepared following the methods described in Example 1.

TABLE I

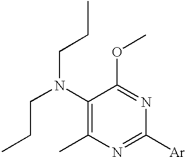

| EX# | Ar | ¹H NMR (CDCl₃) | MS (CI) | Name |
|---|---|---|---|---|
| 2 | 2-chlorophenyl | 0.85(t, 6H), 1.38(m, 4H), 2.58(s, 3H), 2.92(t, | 334 | [2-(2-chlorophenyl)-4-methoxy-6- |
| | | 4)H, 4.05(s, 3H), 7.40(m, 2H), 8.3(d, 1H), 8.4 | | methylpyrimidin-5-yl]dipropylamine |
| | | (br s, 1H) | | |
| 3 | 2,4-dichlorophenyl | 0.82(t, 6H), 1.4(m, 4H), 2.58(s, 3H), 2.9 | 368 | [2-(2,4-dichlorophenyl)-4-methoxy-6- |
| | | (t, 4H), 4.0(s, 3H), 7.24(d, 1H), 7.50(d, | | methylpyrimidin-5-yl]dipropylamine |
| | | 1H), 7.6(d, 1H) | | |
| 4 | 4-chloro-2-methoxyphenyl | 0.85(t, 6H), 1.4(m, 4H), 2.56(t, 4H), 3.85 | 364 | [2-(2-methoxy-4-chlorophenyl)-4- |
| | | (s, 3H), 4.05(s, 3H), 6.9(d, 1H), 7.3(d, | | methoxy-6-methylpyrimidin-5- |
| | | 1H), 7.75(d, 1H) | | yl]dipropylamine |
| 5 | 2-methoxy-4-isopropyl | 0.85(t, 6H), 1.2(d, 6H), 1.4(m, 4H),3.90 | 372 | [2-(2-methoxy-4-isopropylphenyl)-4- |
| | phenyl | (m, 5H), 3.82(s, 3H), 4.00(s, 3H), 6.95(d, | | methoxy-6-methylpyrimidin-5- |
| | | 1H), 7.22(d, 1H), 7.55(d, 1H) | | yl]dipropylamine |
| 6 | 2,4-dimethoxyphenyl | 0.85(t, 6H), 1.4(m, 4H), 2.58(s, 3H), | 360 | [2-(2,4-dimethoxyphenyl)-4-methoxy- |
| | | 2.92(t, 4H), 3.82(s, 3H), 3.85(m 2H), | | 6-methyl pyrimidin-5-yl] |
| | | 4.02(s, 3H), 6.90(d, 1H), 6.95(d, 1H), 7.3 | | dipropylamine |
| | | (d, 1H) | | |
| 6a | 2,6-dimethoxyphenyl | | | [4-Methoxy-2-(2,6-dimethoxy- |
| | | | | phenyl)-6-methyl-pyrimidin-5-yl]- |
| | | | | dipropyl-amine |
| 6b | 2-methoxy-6- | | | [4-Methoxy-2-(2-methoxy-6- |
| | trifluoromethoxyphenyl | | | trifluormethoxy-phenyl)-6-methyl- |
| | | | | pyrimidin-5-yl]-dipropylamine |

### Example 7

[4-methoxy-2-(6-methoxy-2,4-dimethylphenyl)-6-methylpyrimidin-5-yl]dipropylamine [Formula I: Ar=6-methoxy-2,4-dimethylphenyl; R₁=OCH₃; R₂=N(CH₂CH₂CH₃)₂; R₃=CH₃]

A. A solution of 2-chloro-4-methoxy-6-methyl-5-nitropyrimidine (305 mg, 1.5 mmol) and tetrakis(triphenylphosphine)palladium(0) (33 mg; 2 mol %) in ethyleneglycol dimethyl ether (8 mL) is stirred at room temperature for 15 min, then 2,4-dimethyl-6-methoxybenzeneboronic acid (1.76 mmol) and an aqueous solution of sodium carbonate (1.0 M, 4 mL) are added sequentially. The mixture is heated to 75° C. with stirring for 1.5 h, then diluted with 0.1 N sodium hydroxide and extracted twice with 1:1 hexane-ethyl ether. Combined extracts are dried (sodium sulfate), filtered, concentrated and chromatographed on silica (4:1 hexane-ether) to give 3-methoxy-2-(4-methoxy-6-methyl-5-nitropyrimidin-2-yl)- 1,5-dimethylbenzene (0.36 g): ¹H NMR (CDCl₃, 400 MHz) 2.08 (s, 3H), 2.35 (s, 3H), 2.58 (s, 3H), 3.7 (s, 3H), 4.05 (s, 3H), 6.65 (s, 1H), 6.72); MS (CI) 304.

B. To a solution of 3-methoxy-2-(4-methoxy-6-methyl-5-nitropyrimidin-2-yl)- 1,5-dimethylbenzene (1.51 g, 5.00 mmol) in THF (20 mL) and water (20 mL) is added sodium hydrosulfite (8.6 g, 50.0 mmol). The mixture is stirred at room temperature for 14 h, diluted with saturated aqueous sodium bicarbonate, and extracted with ethyl acetate. Combined extracts are washed with brine, dried (sodium sulfate), filtered and concentrated to give 4-methoxy-2-(6- methoxy-2,4-dimethylphenyl)-6-methylpyrimidine-5-ylamine (1.05 g): ¹H NMR (CDCl₃, 400 MHz) 2.05 (s, 3H), 2.34 (s, 3H), 2.38 (s, 3H), 3.60 (br s, 2H), 3.68 (s, 3H), 4.00 (s, 3H), 6,60 (s, 1H), 6.64 (s, 1H).

C. To a solution of 4-methoxy-2-(6-methoxy-2,4-dimethylphenyl)-6-methylpyrimidine-5-ylamine (50 mg, 0.2 mmol) in 1,2-dichloroethane (5 mL) is added propionaldehyde (70 mg, 1.2 mmol) and glacial acetic acid (60 mg). After 10 minutes sodium triacetoxyborohydride (250 mg, 1.2 mmol) is added in one portion. The solution is stirred at room temperature for 1 h and the volatiles removed by rotary evaporation. The residue is partitioned between ethyl acetate and saturated aqueous sodium bicarbonate, the layers are separated and the aqueous layer further extracted with ethyl acetate. The combined organics are washed with water, brine, dried (sodium sulfate), filtered, concentrated and chromatographed on silica (4:1 hexane-ether) to give to give the title compound (56 mg): ¹H NMR (CDCl₃, 400 MHz) δ0.85 (t, 6H), 1.4 (m, 4H), 2.02 (s, 3H), 2.34 (s, 3H), 2.56 (s, 3H), 2.92 (t, 4H), 3.84 (s, 3H), 3.94 (s, 3H), 6.62 (s, 1H), 6.65 (s, 1H); MS (CI) 358.

The EX#s 8-20 in the Table II may be prepared following the methods described in Example 7.

TABLE II

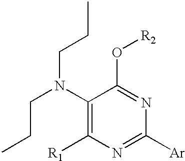

| | | | | | MS | |
|---|---|---|---|---|---|---|
| EX# | Ar | R₁ | R₂ | ¹H NMR (CDCl₃) | (CI) | Name |
| 8 | 2-methoxy-4,6- | Et | Me | 0.85(t, 6H), 1.22(t, 3H), 1.38 | 372 | [2-(2-methoxy-4,6- |
| | dimethylphenyl | | | (m, 4H), 2.05(s, 3H), 2.38(s, | | dimethylphenyl)-4- |
| | | | | 3H), 2.85(m, 6H) 3.75(s, 3H), | | methoxy-6-ethyl |
| | | | | 3.84(s, 3H), 6.62(s, 1H), 6.68 | | pyrimidin-5-yl] |
| | | | | (s, 1H) | | dipropylamine |
| 9 | 2,4,6-trimethylphenyl | Me | Me | 0.80(t, 6H), 1.38(m, 4H), 2.12 | 342 | [2-2,4,6-trimethylphenyl)- |
| | | | | (s, 6H), 2.30(s, 3H), 2.55(s, | | 4-methoxy-6-methyl |
| | | | | 3H), 2.85(m, 4H), 3.95(s, 3H), | | pyrimidin-5-yl] |
| | | | | 6.90(s, 2H) | | dipropylamine |
| 10 | 2,4,6-trimethylphenyl | Et | Me | 0.85(t, 6H), 1.22(t, 3H), 1.40 | 356 | [2-(2,4,6-trimethylphenyl)- |
| | | | | (m, 4H), 2.15(s, 6H), 2.30(s, | | 4-methoxy-6-ethyl |
| | | | | 3H), 2.90(m, 6H), 3.92(s, 3H), | | pyrimidin-5-yl] |
| | | | | 6.9(s, 2H) | | dipropylamine |
| 11 | 2-methoxy-4,6- | Me | Et | 0.89(t, 6H), 1.35(t, 3H), 1.37 | 372 | [2-(2-methoxy-4,6- |
| | dimethylphenyl | | | (m, 4H), 2.05(s, 3H), 2.32(s, | | dimethylphenyl)-4-ethoxy- |
| | | | | 3H), 2.52(s, 3H), 2.94(t, 4H), | | 6-methyl pyrimidin-5-yl] |
| | | | | 3.73(s, 3H), 4.40(q, 2H), 6.62 | | dipropylamine |
| | | | | (s, 1H), 6.66(s, 1H) | | |
| 11a | 2-methoxy-6-trifluoro | Me | Et | | | [2-(2-methoxy-6-trifluoro |
| | | | | | | methoxyphenyl)-4-ethoxy- |
| | | | | | | 6-methyl pyrimidin-5-yl] |
| | | | | | | dipropylamine |
| 12 | 2-methoxy-4,6- | Me | CH₂CH₂F | 0.89(t, 6H), 1.40(m, 4H), 2.04 | 390 | [2-(2-methoxy-4,6- |
| | dimethylphenyl | | | (s, 3H), 2.33(s, 3H), 2.55(s, | | dimethylphenyl)-4-(2- |
| | | | | 3H), 2.96(t, 4H), 3.72(s, 3H), | | fluoroethoxy)-6-methyl |
| | | | | 4.64(m, 2H), 4.66(m, 2H), | | pyrimidin-5-yl] |
| | | | | 6.62(s, 1H), 6.67(s, 1H) | | dipropylamine |
| 13 | 2-methoxy-4,6- | Me | CH(CH₃)₂ | 0.89(t, 6H), 1.31(d, 6H), 1.36 | 344 | [2-(2-methoxy-4,6- |
| | dimethylphenyl | | | (m, 4H), 2.06(s, 3H), 2.33(s, | | dimethylphenyl) |
| | | | | 3H), 2.51(s, 3H), 2.94(t, 4H), | | 4isopropoxy-6-methyl |
| | | | | 3.73(s, 3H), 5.38(hept, 1H), | | pyrimidin-5-yl] |
| | | | | 6.62(s, 1H), 6.67(s, 1H) | | dipropylamine |
| 14 | 2-methoxy-4,6- | CH₂F | Me | 0.88(t,6H), 1.38(m, 4H), 2.07 | 376 | [2-(2-methoxy-4,6- |
| | dimethylphenyl | | | (s, 3H), 2.34(s, 3H), 2.94(t, | | dimethylphenyl)-4- |
| | | | | 4H), 3.73(s, 3H), 3.98(s, 3H), | | methoxy-6-fluoromethyl |
| | | | | 5.60(d, 1H), 6.63(s, 1H), 6.68 | | pyrimidin-5-yl] |
| | | | | (s, 1H) | | dipropylamine |
| 15 | 2-methoxy-4,6- | CHF₂ | Me | 0.89(t, 6H), 1.39(m, 4H), 2.09 | 394 | [2-(2-methoxy-4,6- |
| | dimethylphenyl | | | (s, 3H), 2.34(s, 3H), 2.94(t, | | dimethylphenyl)-4- |
| | | | | 4H), 3.73(s, 3H), 4.01(s, 3H), | | methoxy-6-difluoromethyl |
| | | | | 6.64(s, 1H), 6.68(s, 1H), 7.25 | | pyrimidin-5-yl] |
| | | | | (t, 1H) | | dipropylamine |
| 16 | 2-methoxy-4,6- | —CH(OH)CH₃ | Me | 0.87(t, 6H), 1.27(d, 3H), 1.38 | 402 | 1-[5-(dipropylamino)-6- |
| | dimethylphenyl | | | (m, 4H), 2.18(s, 3H), 2.34(s, | | methoxy-2-(2-methoxy- |
| | | | | 3H), 2.70(dd, 1H), 2.94(t, 4H), | | 4,6-dimethylphenyl)- |
| | | | | 3.34(dd, 1H), 3.75(s, 3H), 3.96 | | pyrimidin-4-yl]-etham-1-ol |
| | | | | (s, 3H), 4.15(m, 1H), 5.70(br | | |
| | | | | s, 1H), 6.64(s, 1H), 6.69(s, | | |
| | | | | 1H) | | |
| 17 | 2-methoxy-4,6- | —C(CH₃)₂OH | Me | 0.88(t, 6H), 1.23(s, 6H), 1.37 | 416 | 1-[5-(dipropylamino)-6- |
| | dimethylphenyl | | | (m, 4H), 2.17(s, 3H), 2.34(s, | | methoxy-2-(2-methoxy- |
| | | | | 3H), 2.90(t, 4H), 3.10(s, 2H), | | 4,6-dimethylphenyl)- |
| | | | | 3.73(s, 3H), 3.96(s, 3H), 6.61 | | pyrimidin-4-yl]-propan-2- |
| | | | | (s, 1H), 6.63(s, 1H), 6.68(s, | | ol |
| | | | | 1H) | | |
| 18 | 2-methoxy-4,6- dimethylphenyl | 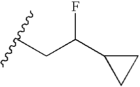 | Me | 0.3-0.6(m, 4H), 0.89(t, 6H), 1.35(t, 3H), 1.41(m, 4H), 2.07 (s, 3H), 2.33(s, 3H), 2.91(t, 4H), 3.20(ddd, 1H), 3.64(ddd, 1H), 3.68(s, 3H), 3.95(s, 3H), 4.40(q, 2H), 4.50(ddt, 1H), 6.64(s, 1H), 6.69(s, 1H) | 430 | [4-(2-Cyclopropyl-2- fluoro-ethyl)-6-methoxy-2- (2-methoxy-4,6-dimethyl- phenyl)-pyrimidin-5-yl]- dipropyl-amine |
| 19 | 2-methoxy-4,6- dimethylphenyl | 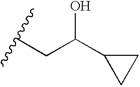 | Me | 0.2-0.6(m, 4H), 0.88(t, 6H), 1.00(m, 1H), 1.41(m, 4H), 2.17(s, 3H), 2.34(s, 3H), 2.91 (t, 4H), 2.92(m, 2H), 3.27(t, 1H), 3.46(d, 1H), 3.74(s, 3H), 3.96(s, 3H), 5.75(br s, 1H), 6.64)(s, 1H), 6.69(s, 1H) | 498 | [4-(2-Cyclopropyl-2- hydroxy-ethyl)-6-methoxy- 2-(2-methoxy-4,6- dimethyl-phenyl)- pyrimidin-5-yl]-dipropyl- amine |
| 20 | 2-methoxy-4,6- dimethylphenyl | 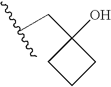 | Me | 0.90(t, 6H), 1.42(m, 4H), 1.37 (m, 4H), 1.61(m, 2H), 1.93(t, 2H), 2.11(m, 2H), 2.14(s, 3H), 2.35(s, 3H), 2.94(t, 4H), 3.26 (s, 1H), 3.74(s, 3H), 3, 96(s, 3H), 6.64(s, 1H), 6.69(s, 1H), 6.78(s, 1H) | 428 | 1-[5-Dipropylamino-6- methoxy-2-(2-methoxy- 4,6-dimethyl-phenyl)- pyrimidin-4-ylmethyl]- cyclobutanol |

### Example 21

(Cyclopropylmethyl)[4-methoxy-2-(6-methoxy-2,4-dimethylphenyl)-6-methylpyrimidin-5-yl]propylamine [Formula I: Ar=6-methoxy-2,4-dimethylphenyl;

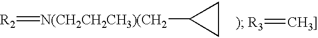

A. To a stirred solution of 4-methoxy-2-(6-methoxy-2,4-dimethylphenyl)-6-methylpyrimidine-5-ylamine (900 mg, 3.3 mmol) in ethyl acetate (15 mL) is added triethylamine (430 mg, 4.25 mmol) and then cyclopropanecarbonyl chloride (416 mg, 4.0 mmol). The solution is stirred at room temperature for 1 h and then saturated aqueous sodium bicarbonate is added. The layers are separated and the aqueous layer further extracted with ethyl acetate. The combined organics are washed with water, brine, dried (sodium sulfate), filtered and concentrated to give cyclopropyl-N-[4-methoxy-2-(6-methoxy-2,4-dimethylphenyl)-6-methlylpyrimidin-5-yl]carboxamide (1.02 g): ¹H NMR (CDCl₃, 400 MHz) δ0.85 (br, 2H), 1.1 (m, 2H), 1.2 (m, 1H),2.05 (s, 3H), 2.34 (s, 3H), 2.38 (s, 3H), 3.65 (s, 3H), 3.98 (s, 3H), 6,60 (s, 1H), 6.64 (s, 1H); MS (CI) 342.

B. To a stirred solution of cyclopropyl-N-[4-methoxy-2-(6-methoxy-2,4-dimethylphenyl)-6-methylpyrimidin-5-yl]carboxamide (115 mg, 0.33 mmol) and 1-iodopropane (85 mg, 0.5 mmol) in DMF (2 mL) is added 60% sodium hydride (40 mg, 1.0 mmol). The mixture is heated to 55° C. with stirring for 2 hr, cooled to room temperature and partitioned between ethyl acetate and saturated aqueous sodium bicarbonate. The layers are separated, the aqueous layer further extracted with ethyl acetate and the combined organics washed with water, brine, dried (sodium sulfate), filtered and concentrated to give cyclopropyl-N-[4-methoxy-2-(6-methoxy-2,4-dimethylphenyl)-6-methylpyrimidin-5-yl]-N-propylcarboxamide. An analytically pure sample is prepared by preparative TLC: ¹H NMR (CDCl₃, 400 MHz) δ0.65 (m, 2H), 0.90 (t, 3H), 1.05 (m, 3H), 1.2 (m, 1H), 1.58 (m, 2H), 2.08 (s, 3H), 2.35 (s, 3H), 2.45 (s, 3H), 3.6 (m, 2H), 3.75 (s, 3H), 3.98 (s, 3H), 6,65 (s, 1H), 6.72 (s, 1H); MS (CI) 384.

C. To a stirred solution of cyclopropyl-N-[4-methoxy-2-(6-methoxy-2,4-dimethylphenyl)-6-methylpyrimidin-5-yl]-N-propylcarboxamide (90 mg, 0.2 mmol) in toluene at room temperature is added diisobutylaluminum hydride 1 M solution in hexanes (0.6 mL, 0.6 mmol). After 1 h the solution is acidified with 1 M hydrochloric acid, neutralized with 2 M sodium hydroxide solution and extracted into ethyl acetate. The combined extracts are washed with water, brine, dried (sodium sulfate), filtered, concentrated, and chromatographed on silica (4:1 hexane-ether) to give 2- {5-[cyclopropylmethyl)propylamino]-4-methoxy-6-methylpyrimidin-2-yl}-3,5-dimethylphenol (61 mg): ¹H NMR (CDCl₃, 400 MHz) δ0.02 (d, 2H), 0.38 (d, 2H), 0.78 (m, 1H), 0.85 (t, 3H), 1.28 (m, 2H), 2.28 (s, 3H), 2.58 (s, 3H), 2.78 (s, 3H), 2.82 (d, 2H), 2.95 (t, 2H), 4.02 (s, 3H), 6,60 (s, 1H), 6.72 (s, 1H) ); MS (CI) 356.

D. To 2- {5-[cyclopropylmethyl)propylamino]-4-methoxy-6-methylpyrimidin-2-yl}-3,5-dimethylphenol (35 mg, 0.1 mmol) in DMF (1 mL) is added cesium carbonate (163 mg, 0.5 mmol) and methyl iodide (0.1 mmol). The mixture is heated to 55° C. with stirring for 2 h, cooled to room temperature and partitioned between ethyl acetate and saturated aqueous sodium bicarbonate. The layers are separated, the aqueous layer further extracted with ethyl acetate and the combined organics washed with water, brine, dried (sodium sulfate), filtered and concentrated. The residue is purified by preparative TLC to give the title compound (22 mg): MS (CI) 370.

The EX#s 22-25 in the Table III may be prepared following the methods described in Example 21

TABLE III

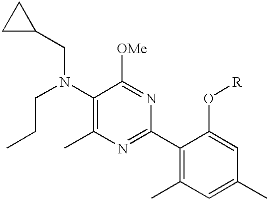

| EX# | R | MS (CI) | NAME |
|---|---|---|---|
| 22 | Et | 384 | Cycloproplmethyl-[2-(2-ethoxy-4,6- |
| | | | dimethylphenyl)-4-methoxy-6-methyl |
| | | | pyrimidin-5-yl]propyl-amine |
| 23 | Pr | 398 | Cyclopropylmethyl[2-(2-propoxy-4,6- |
| | | | dimethylphenyl)-4-methoxy-6- |
| | | | methylpyrimidin-5-yl]dipropylamine |
| 24 | iPr | 398 | Cyclopropylmethyl[2-(2-isopropoxy-4, |
| | | | 6-dimethylphenyl)-4-methoxy-6- |
| | | | methylpyrimidin-5-yl] |
| | | | dipropylamine |
| 25 | CH₂CH₂OMe | 414 | Cyclopropylmethyl[2-(2- |
| | | | ethoxymethoxy-4,6- |
| | | | dimethylphenyl)-4-methoxy-6- |
| | | | methylpyrimidin-5 -yl]dipropylamine |

### Example 26

[2-(dimethylamino)ethyl](cyclopropylmethyl) [6-methoxy-2-(6-methoxy-2,4-dimethiylphenyl)-4-methylpyrimidin-5-yl]amine [Formula I: Ar=6-methoxy-2,4-dimethylphenyl;

A. To a stirred solution of cyclopropyl-N-[4-methoxy-2-(6-methoxy-2,4-dimethylphenyl)-6-methylpyrimidin-5-yl]carboxamide (115 mg, 0.33 mmol) and 2-(dimethylamino)ethyl chloride hydrochloride (72 mg, 0.5 mmol) in DMF (2 mL) is added 60% sodium hydride (40 mg, 1.0 mmol. The mixture is heated to 55° C. with stirring for 2 h, cooled to room temperature and partitioned between ethyl acetate and saturated aqueous sodium bicarbonate. The layers are separated, the aqueous layer further extracted with ethyl acetate and the combined organics washed with water, brine, dried (sodium sulfate), filtered and concentrated to give N-[2-(dimethylamino)ethyl]cyclopropyl-N- [6-methoxy-2-(6-methoxy-2,4-dimethylphenyl)-4-methylpyrimidin-5-yl]carboxamide (121 mg): MS (CI) 413.

B. To a stirred solution of N-[2-(dimethyl amino) ethyl]cyclopropyl-N-[6-methoxy-2-(6-methoxy-2,4-dimethylphenyl)-4-methylpyrimidin-5-yl]carboxamide (82 mg, 0.2 mmol) in tolune at room temperature is added diisobutylaluminum hydride 1 M solution in hexanes (0.4 mL, 0.4 mmol). After 1 h the solution is acidified with 1 M hydrochloric acid, neutralized with 2 M sodium hydroxide solution and extracted into ethyl acetate. The combined extracts are washed with water, brine, dried (sodium sulfate), filtered, concentrated and purified by preparative TLC to give the title compound (61 mg): MS (CT) 399.

EX#s 27-30 in the Table IV may be prepared following the methods described in Example 26.

TABLE IV

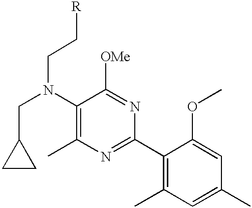

| EX# | R | MS (CI) | NAME |
|---|---|---|---|
| 27 | 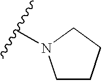 | 425 | Cyclopropylmethyl-[4-methoxy-2- (2-methoxy-4,6-dimethyl-phenyl)- 6-methyl-pyrimidin-5-yl]-(2- pyrrolidi n-1-yl-ethyl)-amine |
| 28 | 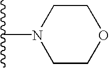 | 442 | Cyclopropylmethyl-[4-methoxy-2- (2-methoxy-4,6-dimethyl-phenyl)- 6-methyl-pyrimidin-5-yl]-(2- morpholin -1-yl-ethyl)-amine |
| 29 | OMe | 400 | Cyclopropylmethyl-(2-methoxy- ethyl)-[4-methoxy-2-(2-methoxy- 4,6-dimethyl-phenyl)-6-methyl- pyrimidin- 5-yl]-amine |
| 30 | 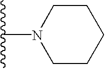 | 440 | Cyclopropylmethyl-[4-methoxy-2- (2-methoxy-4,6-dimethyl-phenyl)- 6-methyl-pyrimidin-5-yl]-(2- piperidin-1-yl-ethyl)-amine |

### Example 31

(Ethylpropyl) [6-methoxy-2-(6-methoxy-2,4-dimethylphenyl)-4-methylpyrimidin-5-yl]amine [Formula I: Ar=6-methoxy-2,4-dimethylphenyl; R₁=CH₃; R₂=N(CH₂CH₃)(CH₂CH₂CH₃); R₃=OCH₃]

A suspension of 4-methoxy-2-(6-methoxy-2,4-dimethylphenyl)-6-methylpyrimidin-5-ylamine (100 mg, 0.37 mmol), Kl (166 mg, 1.0 mmol), K₂CO₃ (138 mg, 1.0 mmol) and 2-bromopentane (151 mg, 1.0 mmol) in anhydrous acetonitrile (5 mL) in a pressure tube is heated at 100° C. for 48 h. After cooling down, the mixture is partitioned between ethyl ether (50 mL) and brine (30 mL). The organic phase is washed with brine (30 mL), dried (MgSO₄) and the solvent removed under reduced pressure. Flash chromatography (25% ethyl acetate in hexanes) yields 6.9 mg of (ethylpropyl)[6-methoxy-2-(6-methoxy-2,4-dimethylphenyl)-4-methylpyrimidin-5-yl]amine. ¹H NMR (CDCl₃, 400 MHz) δ0.95 (t, 6H), 1.49 (m, 4H), 2.04 (s, 3H), 2.32 (s, 3H) 2.46 (s, 3H), 3.27 (m, 1H), 3.71 (s, 3H), 3.95 (s, 3H), 6.62 (s, 1H), 6.67 (s, 1H); MS (CI): 344.

TABLE V

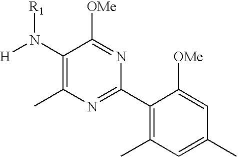

| | | | MS | |
|---|---|---|---|---|
| EX# | R₁ | ¹H NMR (CDC₃) | (CI) | NAME |
| 32 | 1-Propyl | 1.00(t, 3H), 1.59(m, 2H), 2.06 | 368 | [4-Methoxy-2-(2-methoxy-4,6- |
| | | (s, 3H), 2.33(s, 3H), 2.49(s, | | dimethyl-phenyl)-6-methyl- |
| | | 3H), 3.11(t, 2H), 3.72(s, 3H), | | pyrimidin-5-yl]-propyl-amine |
| | | 3.96(s, 3H), 6.62(s, 1H), 6.67 | | |
| | | (s, 1H). | | |
| 33 | Cyclopentyl | 1.45(m, 2H), 1.62(m, 2H), 1.76 | 342 | [4-Methoxy-2-(2-methoxy-4,6- |
| | | (m, 2H), 1.88(m, 2H), 2.05(s, | | dimethyl-phenyl)-6-methyl- |
| | | 3H), 2.32(s, 3H), 2.49(s, 3H), | | pyrimidin-5-yl]-cyclopentyl- |
| | | 3.72 (s, 3H), 3.95(s, 3H), 6.62 | | amine |
| | | (s, 1H), 6.67(2, 1H) | | |
| 34 | —CH₂CN | 2.06(s, 3H), 2.34(s, 3H), 2.52 | 313 | [4-Methoxy-2-(2-methoxy-4,6- |
| | | (s, 3H), 3.64(t, 1H), 3.73(s, 3H), | | dimethyl-phenyl)-6-methyl- |
| | | 4.01(s, 3H), 4.13(d, 2H), 6.63 | | pyrimidin-5-ylamino]- |
| | | (s, 1H), 6.69(s, 1H) | | acetonitrile |

### Example 35

[2-(2-{5-[(cyclopropylmethyl)propylamino]-4-methoxy-6-methylpyrimidin-2-yl}-3,5-dimethylphenoxy)ethyl]dimethylamine [Formula I: Ar=2-(2-dimethylamineethoxy)-3,5-dimethylphenyl;

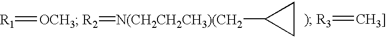

A. To a stirred solution of 2-{5-[cyclopropylmethyl)propylamino]-4-methoxy-6-methylpyrimidin-2-yl}-3,5-dimethylphenol (1.0 g, 2.8 mmol) in DMF (50 mL) is added cesium carbonate (3.26 g, 10.0 mmol) and 2-iodo-1-(1,1,2,2-tetramethy-1-silapropoxy)ethane (1.43 g, 5.0 mmol). The mixture is heated to 80C. with stirring for 2 h, cooled to room temperature and treated with methanol (40 mL) and 2 M hydrochloric acid (60 mL). The mixture is stirred overnight, basified with 4 M sodium hydroxide, extracted into ethyl acetate, washed with water, brine, dried (sodium sulfate), filtered and concentrated to give the crude 2-(2- {5-[(cyclopropylmethyl)propylamino]-4-methoxy-6-methylpyrimidin-2-yl}-3,5-dimethylphenoxy)ethan- 1-ol (910 mg)

B. To the crude 2-(2-{5-[(cyclopropylmethyl)propylamino]-4-methoxy-6-methylpyrimidin-2-yl}-3,5-dimethylphenoxy)ethan- 1-ol (40 mg, 0.1 mmol) in dichloromethane (1 ml) is added triethylamine (22 mg, 0.2 mmol) and methanesulfonyl chloride (22 mg, 0.4 mmol). After 1 hr the volatiles are removed by evaporation and the residue redissolved in acetonitrile (1 mL) and potassium carbonate (27 mg, 0.2 mmol) added followed by 1 M dimethylamine in THF (0.2 mL, 0.2 mmol). The mixture is heated to 60 C. with stirring for 2 h, cooled to room temperature partitioned between ethyl acetate and saturated aqueous sodium bicarbonate. The layers are separated, the aqueous layer further extracted with ethyl acetate and the combined organics washed with water, brine, dried (sodium sulfate), filtered and concentrated. The residue is purified by preparative TLC to give the title compound (36 mg): MS (CI) 427. The EX#s 36-52 in the Table VI may be prepared following the methods described in Example 35.

TABLE VI

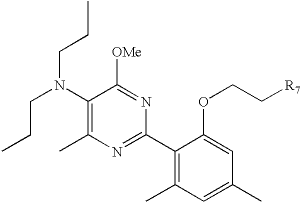

| EX# | R₇ | MS (CI) | NAME |
|---|---|---|---|
| 36 | 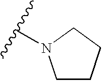 | 454 | {2-[2,4-Dimethyl-6-(2-pyrrolidin-1-yl-ethoxy)- phenyl]-4-methoxy-6-methyl-pyrimidin-5-yl}- dipropyl-amine |
| 37 | 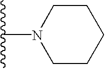 | 468 | {2-[2,4-Dimethyl-6-(2-piperidin-1-yl-ethoxy)- phenyl]-4-methoxy-6-methyl-pyrimidin-5-yl}- dipropylamine |
| 38 | 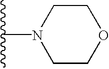 | 470 | {2-[2,4-Dimethyl-6-(2-morpholin-1-yl-ethoxy)- phenyl]-4-methoxy-6-methyl-pyrimidin-5-yl}- dipropyl-amine |
| 39 | 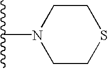 | 486 | {2-[2,4-Dimethyl-6-(2-thiomorpholin-4-yl- ethoxy)-phenyl]-4-methoxy-6-methyl-pyrimidin- 5-yl}-dipropyl-amine |
| 40 | 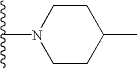 | 482 | (2-{2,4-Dimethyl-6-[2-(4-methyl-piperidin-1-yl)- ethoxy]-phenyl}-4-methoxy-6-methyl-pyrimidin- 5-yl)-dipropyl-amine |
| 41 | 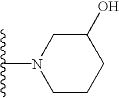 | 484 | (2-{2,4-Dimethyl-6-[2-(3-hydroxy-piperidin-1- yl)-ethoxy]-phenyl}-4-methoxy-6-methyl- pyrimidin-5-yl)-dipropyl-amine |
| 42 | 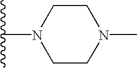 | 483 | (2-{2,4-Dimethyl-6-[2-(4-methyl-piperizin-1-yl)- ethoxy]-phenyl}-4-methoxy-6-methyl-pyrimidin- 5-yl)-dipropyl-amine |
| 43 | 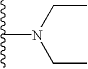 | 456 | {2-[2-(2-Diethylamino-ethoxy)-4,6-dimethyl- phenyl]-4-methoxy-6-methyl-pyrimidin-5-yl}- dipropyl-amine |
| 44 | 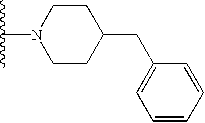 | 558 | (2-{2-[2-(4-Benzyl-piperidin-1-yl)-ethoxy]-4,6- dimethyl-phenyl}-4-methoxy-6-methyl- pyrimidin-5-yl)-dipropyl-amine |
| 45 | 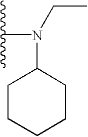 | 510 | (2-{2-[2-(Cyclohexyl-ethyl-amino)-ethoxy]-4,6- dimethyl-phenyl}-4-methoxy-6-methyl- pyrimidin-5-yl)-dipropyl-amine |
| 46 | 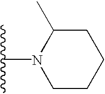 | 482 | (2-{2,4-Dimethyl-6-[2-(2-methyl-piperidin-1-yl)- ethoxy]-phenyl}-4-methoxy-6-methyl-pyrimidin- 5-yl)-dipropyl-amine |
| 47 | 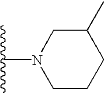 | 482 | (2-{2,4-Dimethyl-6-[2-(3-methyl-piperidin-1-yl)- ethoxy]-phenyl}-4-methoxy-6-methyl-pyrimidin- 5-yl)-dipropyl-amine |
| 48 | 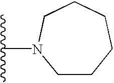 | 482 | {2-[2-(2-Azepan-1-yl-ethoxy)-4,6-dimethyl- phenyl]-4-methoxy-6-methyl-pyrimidin-5-yl}- dipropyl-amine |
| 49 | 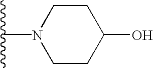 | 484 | (2-{2,4-Dimethyl-6-[2-(4-hydroxy-piperidin-1- yl)-ethoxy]-phenyl}-4-methoxy-6-methyl- pyrimidin-5-yl)-dipropyl-amine |
| 50 | 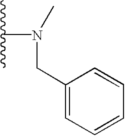 | 504 | (2-{2-[2-(Benzyl-methyl-amino)-ethoxy]-4,6- dimethyl-phenyl}-4-methoxy-6-methyl- pyrimidin-5-yl)-dipropyl-amine |
| 51 | 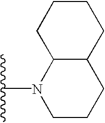 | 522 | (2-{2,4-Dimethyl-6-[2-(octahydro-quinolin-1-yl)- ethoxy]-phenyl}-4-methoxy-6-methyl-pyrimidin- 5-yl)-dipropyl-amine |
| 52 | 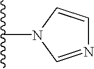 | 451 | {2-[2-(2-Imidazol-1-yl-ethoxy)-4,6-dimethyl- phenyl]-4-methoxy-6-methyl-pyrimidin-5-yl}- dipropyl-amine |

### Example 53

Tert-butyl-N-[4-methoxy-2-(2,6-dimethoxypheny)-6-methylpyrimidin-5-yl]carboxamide may be prepared following the method described in Step A of Example 21 starting from 4-methoxy-2-(2,6-dimethoxypheny)-6-methylpyrimidin-5-yl amine.

### Example 54

Tert-butyl-N-[n-propyl]-N-[4-methoxy-2-(2,6-dimethoxypheny)-6-methylpyrimidin-5-yl]carboxamide may be prepared following the method described in Step B of Example 21 starting from tert-butyl-N-[4-methoxy-2-(2,6-dimethoxypheny)-6-methylpyrimidin-5-yl]carboxamide.

### Additional Examples

The following compounds can be prepared using the methods given in reaction schemes I-IV and further illustrated in the preceding examples.

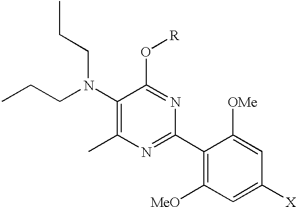

| EX# | X | R |
|---|---|---|
| 55 | OMe | Me |
| 56 | OMe | Et |
| 57 | OMe | OCF₃ |
| 58 | OMe | OCHF₂ |
| 59 | OCF₃ | Me |
| 60 | OCF₃ | Et |
| 61 | OCF₃ | OCF₃ |
| 62 | OCF₃ | OCHF₂ |
| 63 | OCHF₂ | Me |
| 64 | OCHF₂ | Et |
| 65 | OCHF₂ | OCF₃ |
| 66 | OCHF₂ | OCHF₂ |
| 67 | Cl | Me |
| 68 | Cl | Et |
| 69 | Cl | OCF₃ |
| 70 | Cl | OCHF₂ |
| 80 | F | Me |
| 81 | F | Et |
| 82 | F | OCF₃ |
| 83 | F | OCHF₂ |
| 84 | Me | Me |
| 85 | Me | Et |
| 86 | Me | OCF₃ |
| 87 | Me | OCHF₂ |
| 88 | Et | Me |
| 89 | Et | Et |
| 90 | Et | OCF₃ |
| 91 | Et | OCHF₂ |
| 92 | CF₃ | Me |
| 93 | CF₃ | Et |
| 94 | CF₃ | OCF₃ |
| 95 | CF₃ | OCHF₂ |

### Example 96

Assay for CRF Receptor Binding Activity

As discussed above, the following assay is defined herein as a standard in vitro CRF receptor binding assay.

The pharmaceutical utility of compounds of this invention is indicated by the following assay for CRF1 receptor activity. The CRF receptor binding is performed using a modified version of the assay described by Grigoriadis and De Souza (Methods in Neurosciences, Vol. 5, 1991). IMR-32 human neuroblastoma cells, a cell-line that naturally expresses the CRF1 receptor, are grown to confluency in DMEM containing FBS.

To prepare receptor containing membranes cells are homogenized in wash buffer (50 mM Tris HCl, 10 mM MgCl₂, 2 mM EGTA, pH 7.4) and centrifuged at 48,000×g for 10 minutes at 4° C. The pellet is re-suspended in wash buffer and the homogenization and centrifugation steps are performed two additional times.

Membrane pellets containing CRF receptors are re-suspended in 50 mM Tris buffer pH 7.7 containing 10 mM MgCl₂ and 2 mM EDTA and centrifuged for 10 minutes at 48000 g. Membranes are washed again and brought to a final concentration of 1500 mg/ml in binding buffer (Tris buffer above with 0.1% BSA, 15 mM bacitracin and 0.01 mg/ml aprotinin.). For the binding assay, 100 ml of the membrane preparation are added to 96 well microtube plates containing 100 ml of ¹²⁵I-CRF (SA 2200 Ci/mmol, final concentration of 100 pM) and 50 ml of test compound. Binding is carried out at room temperature for 2 hours. Plates are then harvested on a Brandel 96 well cell harvester and filters are counted for gamma emissions on a Wallac 1205 Betaplate liquid scintillation counter. Non specific binding is defined by 1 mM cold CRF. IC₅₀ values are calculated with the non-linear curve fitting program RS/1 (BBN Software Products Corp., Cambridge, Mass.). The binding affinity for the compounds of Formula I expressed as IC₅₀ value, generally ranges from about 0.5 nanomolar to about 10 micromolar. Preferred compounds of Formula I exhibit IC₅₀ values of less than or equal to 1.5 micromolar, more preferred compounds of Formula I exhibit IC₅₀ values of less than 500 nanomolar, still more preferred compounds of Formula I exhibit IC50 values of less than 100 nanomolar, and most preferred compound of Formula I exhibit IC₅₀ values of less than 10 nanomolar. The compounds shown in Examples 1-54 have been tested in this assay and found to exhibit IC₅₀ values of less than or equal to 4 micromolar.

### Example 97

Preparation of radiolabeled probe compounds of the invention

The compounds of the invention are prepared as radiolabeled probes by carrying out their synthesis using precursors comprising at least one atom that is a radioisotope. The radioisotope is preferably selected from of at least one of carbon (preferably ¹⁴C), hydrogen (preferably ³H), sulfur (preferably ³⁵S), or iodine (preferably ¹²⁵I). Such radiolabeled probes are conveniently synthesized by a radioisotope supplier specializing in custom synthesis of radiolabeled probe compounds. Such suppliers include Amersham Corporation, Arlington Heights, Ill.; Cambridge Isotope Laboratories, Inc. Andover, Mass.; SRI International, Menlo Park, Calif.; Wizard Laboratories, West Sacramento, Calif.; ChemSyn Laboratories, Lexena, Kans.; American Radiolabeled Chemicals, Inc., St. Louis, Mo.; and Moravek Biochemicals Inc., Brea, Calif.

Tritium labeled probe compounds are also conveniently prepared catalytically via platinum-catalyzed exchange in tritiated acetic acid, acid-catalyzed exchange in tritiated tri fluoroacetic acid, or heterogeneous-catalyzed exchange with tritium gas. Such preparations are also conveniently carried out as a custom radiolabeling by any of the suppliers listed in the preceding paragraph using the compound of the invention as substrate. In addition, certain precursors may be subjected to tritium-halogen exchange with tritium gas, tritium gas reduction of unsaturated bonds, or reduction using sodium borotritide, as appropriate.

### Example 98

Receptor autoradiography

Receptor autoradiography (receptor mapping) is carried out in vitro as described by Kuhar in sections 8.1.1 to 8.1.9 of Current Protocols in Pharmacology (1998) John Wiley & Sons, New York, using radiolabeled compounds of the invention prepared as described in the preceding Examples.

### Example 99

Additional Aspects of Preferred Compounds of the Invention

The most preferred compounds of the invention are suitable for pharmaceutical use in treating human patients. Accordingly, such preferred compounds are non-toxic. They do not exhibit single or multiple dose acute or long-term toxicity, mutagenicity (e.g., as determined in a bacterial reverse mutation assay such as an Ames test), teratogenicity, tumorogenicity, or the like, and rarely trigger adverse effects (side effects) when administered at therapeutically effective dosages.

Preferably, administration of such preferred compounds of the invention at certain doses (e.g., doses yielding therapeutically effective in vivo concentrations or preferably doses of 10, 50, 100, 150, or 200 mg/kg—preferably 150 mg/kg—administered parenterally or prefrerably orally) does not result in prolongation of heart QT intervals (i.e., as determined by electrocardiography, e.g., in guinea pigs, minipigs or dogs). When administered daily for 5 or preferably ten days, such doses of such preferred compounds also do not cause liver enlargement resulting in an increase of liver to body weight ratio of more than 100%, preferably not more than 75% and more preferably not more than 50% over matched controls in laboratory rodents (e.g., mice or rats). In another aspect such doses of such preferred compounds also preferably do not cause liver enlargement resulting in an increase of liver to body weight ratio of more than 50%, preferably preferably not more than 25% and more preferably not more than 10% over matched untreated controls in dogs or other non-rodent animals.

In yet another aspect such doses of such preferred compounds also preferably do not promote the release of liver enzymes (e.g., ALT, LDH, or AST) from hepatocvtes in vivo. Preferably such doses do not elevate such enzymes by more than 100%, preferably not by more than 75% and more preferably not by more than 50% over matched untreated controls in laboratory rodents. Similarly, concentrations (in culture media or other such solutions that are contacted and incubated with cells in vitro) equivalent to two, fold, preferably five-fold, and most preferably ten-fold the minimum in vivo therapeutic concentration do not cause release of any of such liver enzymes from hepatocytes in vitro.

Because side effects are often due to undesirable receptor activation or antagonism, preferred compounds of the invention exert their receptor-modulatory effects with high selectivity. This means that they do not bind to certain other receptors (i.e., other than CRF receptors) with high affinity, but rather only bind to, activate, or inhibit the activity of such other receptors with affinity constants of greater than 100 nanomolar, preferably greater than 1 micromolar, more preferably greater than 10 micromolar and most preferably greater than 100 micromolar. Such receptors preferably are selected from the group including ion channel receptors, including sodium ion channel receptors, neurotransmitter receptors such as alpha- and beta-adrenergic receptors, muscarinic receptors (particularly m1, m2, and m3 receptors), dopamine receptors, and metabotropic glutamate receptors; and also include histamine receptors and cytokine receptors, e.g., interleukin receptors, particularly IL-8 receptors. The group of other receptors to which preferred compounds do not bind with high affinity also includes GABAA receptors, bioactive peptide receptors (including NPY and VIP receptors), neurokinin receptors, bradykinin receptors (e.g., BK1 receptors and BK2 receptors), and hormone receptors (including thyrotropin releasing hormone receptors and melanocyte-concentrating hormone receptors).

### Example 99a

Absence of Sodium Ion Channel Activity

Preferred compounds of the invention do not exhibit activity as Sodium ion channel blockers. Sodium channel activity may be measured a standard in vitro sodium channel binding assays such as the assay given by Brown et al. (J. Neurosci. (1986) 265: 17995-18004). Preferred compounds of the invention exhibit less than 15 percent inhibition, and more preferably less than 10 percent inhibition, of sodium channel specific ligand binding when present at a concentration of 4 uM. The sodium ion channel specific ligand used may be labeled batrachotoxinin, tetrodotoxin, or saxitoxin. Such assays, including the assay of Brown referred to above, are performed as a commercial service by CEREP, INC., Redmond, Wash.

Alternatively, sodium ion channel activity may be measured in vivo in an assay of anti-epileptic activity. Anti-epileptic activity of compounds may be measured by the ability of the compounds to inhibit hind limb extension in the supra maximal electro shock model. Male Han Wistar rats (150-200mg) are dosed i.p. with a suspension of 1 to 20 mg of test compound in 0.25% methylcellulose 2 hr. prior to test. A visual observation is carried out just prior to testing for the presence of ataxia. Using auricular electrodes a current of 200 mA, duration 200 millisec, is applied and the presence or absence of hind limb extension is noted. Preferred compounds of the invention do not exhibit significant anti-epileptic activity at the p<0.1 level of significance or more preferably at the p<0.05 level of significance as measured using a standard parametric assay of statistical significance such as a student's T test.

### Example 99b

Optimal in vitro half-life

Compound half-life values (t_{½} values) may be determined via the following standard liver microsomal half-life assay. Liver microsomes obtained from pooled liver samples and prepared so that the P-450 enzyme content is approximately 0.5 mmol/mg protein. Reactions are preformed in a 5 ml well deep-well plate as follows: Phosphate buffer: 19 mL 0.1 M NaH₂PO₄, 81 mL 0.1 Na₂HPO₄, pH 7.4 with H₃PO₄. CoFactor Mixture: 16.2 mg NADP, 45.4 mg Glucose-6-phosphate in 4 mL 100 mM MgCl₂. Glucose-6-phosphate dehydrogenase: 214.3 ul glucose-6-phosphate dehydrogenase, 1285.7 ul distilled water Starting Reaction Mixture: 3 mL CoFactor Mixture, 1.2 mL Glucose-6-phosphate dehydrogenase 6 identical samples wells each containing 25 ul microsomes, 5 ul of test compound (from a 100 uM stock), and 399 ul 0.1 M phosphate buffer, pH 7.4, are prepared. A seventh well containing 25 ul microsomes, 399 ul 0.1 M phosphate buffer, pH 7.4, and 5 ul (from a 100 uM stock) of a compound, e.g. DIAZEPAM, CLOZEPINE, with known metabolic properties is used as a positive control. Reactions are preincubated at 39° C. for 10 minutes. 71 ul Starting Reaction Mixture is added to 5 of the 6 reaction wells and to the positive control well, 71 ul 100 mM MgCl₂ is added to the sixth reaction well, which is used as a negative control. At each time point (0, 1, 3, 5, and 10 minutes) 75 ul reaction is pipetted into a 96-well deep-well plate reaction well containing 75 ul ice-cold acetonitrile. Samples are vortexed and centrifuged 10 minutes at 6000 rpm (Sorval T 6000D rotor). Supernatant, 75 ul from each reaction well, is transferred to a 96-well plate containing 150 ul internal standard per well. The remaining test compound is quantitated via LCMS and Compound concentration vs time is plotted and commercially available statistical software is used to extrapolate to the t_{½} value of the test compound.

Preferred compounds of the invention exhibit in vitro t_{½} values of greater than 10 minutes and less than 4 hours. Most preferred compounds of the invention exhibit in vitro t_{½} values of between 30 minutes and 1 hour in human liver microsomes.

### Example 99c

MDCK Toxicity

Toxicity of a test compound may be assessed by measuring the effect of the compound on ATP production by MDCK cells.

MDCK cells, product no. CCL-34, are purchased from ATCC, Manassas, Va. and maintained in sterile conditions by the methods described in the suppliers production information sheet. The PACKARD BIOSCIENCE, (Groningen, The Netherlands) ATP-LITE-M Luminescent ATP detection kit, product no. 6016941, may be used to monitor ATP production in MDCK cells.

Prior to assay 1 μl of test compound or control sample is pipetted into PACKARD (Meriden, Conn.) clear bottom 96-well plates. Test compounds and control samples are diluted in DMSO to give final concentration in the assay of 10 micromolar, 100 micromolar, or 200 micromolar. Control samples are drug compounds having known toxicity properties.

Confluent MDCK cells are trypsinized, harvested, and diluted to a concentration of 0.1×10⁶ cells/ ml with warm ATCC Eagles Minimum Essential Medium (catalog # 30-2003). Warm Eagle's MEM without cells (100ul) is pipetted in five wells a 96-well plate. These wells are used to determine the standard curve. Cells in Eagles MEM (100μl or 10,000 cells) are pipetted into the remaining wells of the 96-well plates. All samples are incubated at 37° C. under carbogen (95% O₂, 5% CO₂) for 2 hours with constant shaking. After incubation 50 ul mammalian cell lysis solution is added to well of the 96-well plates, wells are covered with Packard topseal stickers, and plates are shaken at approximately 700 rpm on a microtiter shaker for 2 minutes.

During the incubation, Packard ATP Lite-M reagents are allowed to equilibrate to room temperature. Once equilibrated the lyophilized substrate solution in reconstituted in 5.5 mls of substrate buffer solution (from kit). Lyophilized ATP standard solution is reconstituted in deionized water to give a 10 mM stock. Standard (10 ul), diluted so that a 10 ul aliquot yields a final concentration of 200 nM, 100 nM, 50 nM, 25 nM, or 12.5 nM is added to each of the five standard curve wells, which do not contain cells.

Substrate solution (50 ul) is added to all wells. Wells are covered with Packard topseal stickers, and plates are shaken at approximately 700 rpm on a microtiter shaker for 2 minutes. A white Packard sticker is attached to the bottom of each plate and samples are dark adapted by wrapping plates in foil and placing in the dark for 10 minutes. Luminescence is then quantitated at 22° C. using a luminescence counter, e.g. Packard TopCount Microplate Scintillation and Luminescense Counter or Tecan Spectrafluor plus. Luminescence values at each concentration are compared to the values computed from the standard curve for that concentration. Preferred test compounds exhibit luminescence values 80% or more of the standard, or preferably 90% or more of the standard, when 10 micromolar concentration of the test compound is used. When a 100 micromolar concentration of the test compound is used, preferred test compounds exhibit luminescence values 50% or more of the standard, or more preferably 80% or more of the standard.

The disclosures of all articles and references mentioned in this application, including patents, are incorporated herein by reference.

The invention and the manner and process of making and using it, are now described in such full, clear, concise and exact terms as to enable any person skilled in the art to which it pertains, to make and use the sarne. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

What is claimed is:

1. A compound of the formula: 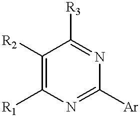
or a pharmaceutically acceptable salt thereof, wherein:
Ar is phenyl, 1- or 2-naphthyl, each of which is mono-, di-, or tri-substituted or mono-, di-, or tri-substituted heteroaryl having from about 5 to about 7 ring members and 1 to about 4 heteroatoms in the ring, the heteroatoms independently selected from the group consisting of N, O and S;
R₁ and R₃ are independently chosen from hydrogen, halogen, cyano, nitro, optionally substituted alkyl, optionally substituted alkenyl, optionally substituted alkynyl, optionally substituted alkoxy, optionally substituted (cycloalkyl)alkyl, optionally substituted alkylthio, optionally substituted alkylsulfinyl, optionally substituted alkylsulfonyl, or optionally substituted mono- or dialkylcarboxamide, with the proviso that R₁ and R₃ are not both hydrogen; and
R₂ is optionally substituted alkyl, optionally substituted alkenyl, optionally substituted alkynyl, optionally substituted alkoxy, optionally substituted aminoalkyl, optionally substituted mono or dialkylamino, optionally substituted alkylthio, optionally substituted alkylsulfinyl, optionally substituted alkylsulfonyl, optionally substituted mono or dialkylcarboxamide, optionally substituted carbocyclic aryl or optionally substituted heteroaryl having from 1 to 3 rings, and 3 to 8 ring members in each ring and 1 to about 3 heteroatoms.

2. A compound of the formula: 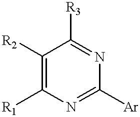
or a pharmaceutically acceptable salt thereof, wherein:
Ar is phenyl which is mono-, di-, or tri-substituted;
R₁ and R₃ are independently chosen from hydrogen, halogen, cyano, nitro, optionally substituted alkyl, optionally substituted alkenyl, optionally substituted alkynyl, optionally substituted alkoxy, optionally substituted (cycloalkyl)alkyl, optionally substituted alkylthio, optionally substituted alkylsulfinyl, or optionally substituted alkylsulfonyl, and optionally substituted mono or dialkylcarboxamide, with the proviso that R₁ and R₃ are not both hydrogen; and
R₂ is optionally substituted alkyl, optionally substituted alkoxy, optionally substituted aminoalkyl, optionally substituted mono or dialkylamino, optionally substituted alkylthio, optionally substituted alkylsulfinyl, optionally substituted alkylsulfonyl, optionally substituted mono or dialkylcarboxamide, or
R₂ is selected from the group consisting of phenyl, naphthyl, pyridyl, pyrimidinyl, pyridizinyl, and thiophenyl, each of which is optionally mono-, di-, or tri-substituted.

3. A compound of the formula 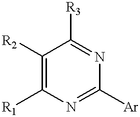
or a pharmaceutically acceptable salt thereof, wherein:
R₁ and R₃ are independently selected from hydrogen, halogen, cyano, C₁₋₆alkyll, (C₃₋₇cycloalkyll) C₁₋₄alkyll, —O(C₃₋₇cycloalkyl,)C₁₋₄alkyll, halo(C₁₋₆)alkyll, —O(halo(C₁₋₆)alkyl₁), —O(C₁₋₆alkyl₁), and S(O)ₙ(C₁₋₆alkyl₁), where each alkyl, is independently straight, branched, or cyclic, may contain 1 or more double or triple bonds, and is optionally substituted with one or more substituents independently chosen from halogen, hydroxy, oxo, cyano, C₁₋₄alkoxy, amino, and mono- or di(C₁₋₄)alkylamino, and where each C₃₋₇cycloalkyll is optionally substituted by one or more substituents independently chosen from halogen, hydroxy, oxo, cyano, C₁₋₄alkoxy, amino, and mono- or di(C₁₋₄)alkylamino,
with the proviso that not both R₁ and R₃ are hydrogen;
R₂ is selected from the group consisting of −XRA and Y; and
Ar is selected from the group consisting of phenyl, naphthyl, pyridyl, pyrimidinyl, pyridizinyl, and thiophenyl, each of which is mono-, di-, or tri-substituted with R_{C};
R_{A} and R_{B}, which may be the same or different, are independently selected at each occurrence from: hydrogen and straight, branched, or cyclic alkyl groups, including (cycloalkyl)alkyl groups consisting of 1 to 8 carbon atoms, which straight, branched, or cyclic alkyl groups may contain one or more double or triple bonds, each of which 1 to 8 carbon atoms may be further substituted with one or more substituent(s) independently selected from oxo, hydroxy, halogen, cyano, amino, C₁₋₆alkoxy, —NH(C₁₋₆alkyl), —N(C₁₋₆alkyl)(C₁₋₆alkyl), —NHC(═O)(C₁₋₆alkyl), —N(C₁₋₆alkyl)C(═O)(C₁₋₆alkyl), —NHS(O)ₙ(C₁₋₆alkyl), —S(O)ₙ(C₁₋₆alkyl), —S(O)ₙNH(C₁₋₆alkyl), —S(O)ₘN(C₁₋₆alkyl)(C₁₋₆alkyl), and Z;
R_{C} is independently selected at each occurrence from halogen, cyano, halo(C₁₋₆)alkyl, halo(C₁₋₆)alkoxy, hydroxy, amino, C₁₋₆alkyl substituted with 0-2 R_{D}, C₂₋₆alkenyl substituted with 0-2 R_{D}, C₂₋₆alkynyl substituted with 0-2 R_{D}, C₃₋₇cycloalkyl substituted with 0-2 R_{D}, (C₃₋₇cycloalkyl)C₁₋₄alkyl substituted with 0-2 R_{D}, C₁₋₆alkoxy substituted with 0-2 R_{D}, —NH(C₁₋₆alkyl) substituted with 0-2 R_{D}, —N(C₁₋₆alkyl) (C₁₋₆alkyl) each C₁₋₆alkyl independently substituted with 0-2 R_{D}, −XRA, and Y;
R_{D} is independently selected at each occurrence from the group consisting of halogen, hydroxy, cyano, amino, C₁₋₄alkyl, —O(C₁₋₄alkyl), —NH(C₁₋₄alkyl), —N(C₁₋₄alkyl)(C₁₋₄alkyl), —S(O)ₙ(alkyl), halo(C₁₋₄)alkyl, halo(C₁₋₄)alkoxy, CO(C₁₋₄alkyl), CONH(C₁₋₄alkyl), CON(C₁₋₄alkyl)(C₁₋₄alkyl), −XRA, and Y;
X is independently selected at each occurrence from the group consisting of —CH₂—, —CHR_{B}—, —O—, —C(═O)—, —C(═O)O—, —S(O)ₙ—, —NH—, —NR_{B}—, —C(═O)NH—, —C(═O)NR_{B}—, —S(O)ₙNH—, —S(O)ₙNR_{B}—, —OC(═S)S—, —NHC(═O)—, —NR_{B}C(═O)—, —NHS(O)ₙ—, —OSiHₙ(C₁₋₄alkyl₂₋ₙ—, and —NR_{B}S(O)ₙ—;
Y and Z are independently selected at each occurrence from: 3- to 7-membered carbocyclic or heterocyclic groups which are saturated, unsaturated, or aromatic, which may be further substituted with one or more substituents independently selected from halogen, oxo, hydroxy, amino, cyano, C₁₋₄alkyl, —O(C₁₋₄alkyl), —NH(C₁₋₄alkyl), —N(C₁₋₄alkyl) (C₁₋₄alkyl),and —S(O)ₙ(alkyl),
said 3- to 7-membered heterocyclic groups containing one or more heteroatom(s) independently selected from N, O, and S, with the point of attachment being either carbon or nitrogen; and
n is independently selected at each occurrence from 0, 1, and 2.

4. A compound or salt according to claim 1 wherein
Ar is mono-, di-, or trisubstituted phenyl; and
R₂ is selected from optionally substituted alkoxy, optionally substituted aminoalkyl, and optionally substituted mono or dialkylamino.

5. A compound or salt according to claim 3, wherein:
Ar is phenyl mono-, di-, or tri-substituted with R_{C}.

6. A compound or salt according to claim 3, wherein:
Ar is phenyl mono-, di-, or tri-substituted with R_{C}; and
R₁ and R₃ are independently selected from the group consisting of halogen,
C₁₋₃alkyl, C₁₋₃alkoxy, (C₃₋₇cycloalkyl)C₁₋₃alkyl, (C₃₋₇cycloalkyl) C₁₋₃alkoxy, each of which is unsubstituted or substituted by 1-3 groups independently chosen from hydroxy, amino, cyano, and halogen.

7. A compound or salt according to claim 3, wherein:
Ar is phenyl mono-, di-, or tri-substituted with R_{C}; and
R_{A} and R_{B}, which may be the same or different, are independently selected at each occurrence from: straight, branched, or cyclic alkyl groups having from 1 to 8 carbon atoms, which alkyl groups may contain one or more double or triple bonds.

8. A compound or salt according to claim 3, wherein:
Ar is phenyl mono-, di-, or tri-substituted with R_{C};
R_{A} and R_{B}, which may be the same or different, are independently selected at each occurrence from: straight, branched, or cyclic alkyl groups having from 1 to 8 carbon atoms, which alkyl groups may contain one or more double or triple bonds; and R₁ and R₃ are independently selected from the group consisting of halogen, C₁₋₃alkyl, C₁₋₃alkoxy, (C₃₋₇cycloalkyl)C₁₋₃alkyl, (C₃₋₇cycloalkyl) C₁₋₃alkoxy, each of which is unsubstituted or substituted by 1-3 groups independently chosen from hydroxy, amino, cyano, and halogen.

9. A compound of Formula A 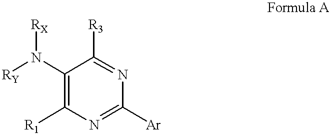
or a pharmaceutically acceptable salt thereof, wherein: R_{X} and R_{Y} are the same or different and are independently selected from:
a) hydrogen,
b) —(C═O)alkyl_{A}, wherein alkyl_{A} is a straight or branched alkyl group having from 1 to 8 carbon atoms;
c) straight, branched, or cyclic alkyl groups, including cycloalkyl(alkyl)groups, said alkyl groups having from 1 to 8 carbon atoms and optionally containing one or more double or triple bonds, each of which alkyl groups may be further substituted with one or more substituent(s) independently selected from:
i) hydroxy, halogen, amino, cyano, —O(C₁₋₄alkyl), —NH(C₁₋₄alkyl), and —NH(C₁₋₄alkyl)(C₁₋₄alkyl), and
ii) 3- to 7-membered carbocyclic and heterocyclic groups, which are saturated, unsaturated, or aromatic, which may be substituted with one or more substituents independently selected from halogen, halo(C₁₋₄)alkyl, halo(C₁₋₄)alkoxy, oxo, hydroxy, amino, C₁₋₄alkyl, —O(C₁₋₄alkyl), —NH(C₁₋₄alkyl), —N(C₁₋₄alkyl)(C₁₋₄alkyl), and —S(O)ₙ(alkyl), wherein said 3- to 7-membered heterocyclic groups contain one or more heteroatom(s) independently selected from N, O, and S, with the point of attachment being either carbon or nitrogen,
R₁ and R₃ are independently selected from hydrogen, halogen, cyano, C₁₋₆alkyll, (C₃₋₇cycloalkyl,) C₁₋₄alkyll, —O(C₃₋₇cycloalkyl,)C₁₋₄alkyll, halo(C₁₋₆)alkyll, —O(halo(C₁₋₆)alkyl₁), —O(C₁₋₆alkyll), and S(O)ₙ(C₁₋₆alkyll), where each said alkyl₁ is straight, branched, or cyclic and may contain 1 or more double or triple bonds, and is optionally substituted by one or more substituents independently chosen from halogen, hydroxy, oxo, cyano, C₁₋₄alkoxy, amino, and mono- or di(C₁₋₄)alkylamino, and where said C₃₋₇cycloalkyll is optionally substituted by one or more substituents independently chosen from halogen, hydroxy, oxo, cyano, C₁₋₄alkoxy, amino, and mono- or di(C₁₋₄)alkylamino
with the proviso that not both R₁ and R₃ are hydrogen;
Ar is selected from the group consisting of phenyl, naphthyl, pyridyl, pyrimidinyl, and thiophenyl, each of which is mono-, di-, or tri-substituted with R_{C};
R_{A} and R_{B}, which may be the same or different, are independently selected at each occurrence from the group consisting of: hydrogen and straight, branched, or cyclic alkyl groups, including (cycloalkyl)alkyl groups, consisting of 1 to 8 carbon atoms, which may contain one or more double or triple bonds, each of which may be further substituted with one or more substituent(s) independently selected from oxo, hydroxy, halogen, nitro, cyano, C₁₋₆alkoxy, —NH(C₁₋₆alkyl), —N(C₁₋₆alkyl)(C₁₋₆alkyl), —NHC(═O)(C₁₋₆alkyl), —N(C₁₋₆alkyl)C(═O)(C₁₋₆alkyl), —NHS(O)ₙ(C₁₋₆alkyl), —S(O)ₙ(C₁₋₆alkyl), —S(O)ₙNH(C₁₋₆alkyl), —S(O)ₙN(C₁₋₆alkyl)(C₁₋₆alkyl), and Z;
R_{C} is independently selected at each occurrence from halogen, cyano, halo(CI-₆)alkyl, halo(C₁₋₆)alkoxy, hydroxy, amino, and C₁₋₆alkyl substituted with 0-2 R_{D}, C₂₋₆alkenyl substituted with 0-2 R_{D}, C₂₋₆alkynyl substituted with 0-2 R_{D}, C₃₋₇cycloalkyl substituted with 0-2 R_{D}, (C₃₋₇cycloalkyl)C₁₋₄alkyl substituted with 0-2 R_{D}, C₁₋₆alkoxy substituted with 0-2 R_{D}, —NH(C₁₋₆alkyl) substituted with 0-2 R_{D}, —N(C₁₋₆alkyl)(C₁₋₆alkyl) each C₁₋₄alkyl independently substituted with 0-2 R_{D}, −XRA, and Y, with the proviso that at least one of the positions ortho or para to the point of attachment of Ar to the pyrimdine ring shown in Formula A is substituted;
R_{D} is independently selected at each occurrence the group consisting of halogen, hydroxy, cyano, C₁₋₄alkyl, —O(C₁₋₄alkyl), —NH(C₁₋₄alkyl), —N(C₁₋₄alkyl)(C₁₋₄alkyl), —S(O)ₙ(alkyl) halo(C₁₋₄)alkyl, halo(C₁₋₄)alkoxy, CO(C₁₋₄alkyl), CONH(C₁₋₄alkyl), CON(C₁₋₄alkyl)(C₁₋₄alkyl), −XRA, and Y;
X is independently selected at each occurrence from the group consisting of —CH₂—, —CHR_{B}—, —O—, —C(═O)—, —C(═O)O—, —S(O)ₙ—, —NH—, —NR_{B}—, —C(═O)NH—, —C(═O)NR_{B}—, —S(O)ₙNH—, —S(O)ₙNR_{B}—, —OC(═S)S—, —NHC(═O)—, —NR_{B}C(═O)—, —NHS(O)ₙ—, —OSiHₙ(C₁₋₄-alkyl₂₋ₙ)—, and —NR_{B}S(O)ₙ—;
Y and Z are independently selected at each occurrence from the group consisting of: 3- to 7-membered carbocyclic and heterocyclic groups, which are saturated, unsaturated, or aromatic, which may be further substituted with one or more substituents independently selected from halogen, oxo, hydroxy, amino, C₁₋₄alkyl, —O(C₁₋₄alkyl), —NH(C₁₋₄alkyl), —N(C₁₋₄alkyl)(C₁₋₄alkyl), and —S(O)ₙ(alkyl); and n is0, 1,or2.

10. A compound or salt according to claim 9, wherein:
R_{X} and R_{Y} are the same or different and are independently selected from:
a) —(C═O)alkyl_{A}, wherein alkyl_{A} is a straight or branched alkyl group having from 1 to 8 carbon atoms;
b) straight, branched, or cyclic alkyl groups, including cycloalkyl(alkyl)groups, said alkyl groups having from 1 to 8 carbon atoms and optionally containing one or more double or triple bonds, each of which alkyl groups may be further substituted with one or more substituent(s) independently selected from:
i) hydroxy, halogen, amino, cyano, —O(C₁₋₄alkyl), —NH(C₁₋₄alkyl), and —NH(C₁₋₄alkyl)(C₁₋₄alkyl), and
ii) 3- to 7-membered carbocyclic and heterocyclic groups, which are saturated, unsaturated, or aromatic, which may be substituted with one or more substituents independently selected from halogen, halo(C₁₋₄)alkyl, halo(C₁₋₄)alkoxy, oxo, hydroxy, amino, C₁₀₄alkyl, —O(C₁₋₄alkyl), —NH(C₁₋₄alkyl), —N(C₁₋₄alkyl)(C₁₋₄alkyl), and —S(O)ₙ(alkyl), wherein said 3- to 7-membered heterocyclic groups contain one or more heteroatom(s) independently selected from N, O, and S, with the point of attachment being either carbon or nitrogen,
R₁ and R₃ are independently selected from C₁₋₆alkyll, (C₃₋₇cycloalkyl₁)C₁₋₄alkyll, —O(C₃₋₇cycloalkyl,) C₁₋₄alkyll, halo(C₁₋₆)alkyl₁, —O(halo(C₁₋₆)alkyl₁), and —O(C₁₋₆alkyl₁), where each said alkyl₁ is straight, branched, or cyclic and may contain 1 or more double or triple bonds, and is optionally substituted by one or more substituents independently chosen from halogen, hydroxy, oxo, cyano, C₁₋₄alkoxy, amino, and mono- or di(C₁₋₄)alkylamino, and where said C₃₋₇cycloalkyl₁ is optionally substituted by one or more substituents independently chosen from halogen, hydroxy, oxo, cyano, C₁₋₄alkoxy, amino, and mono- or di(C₁₋₄)alkylamino
Ar is phenyl, which is mono-, di-, or tri-substituted with R_{C};
R_{A} and R_{B}, which may be the same or different, are independently selected at each occurrence from the group consisting of: hydrogen and straight, branched, or cyclic alkyl groups, including (cycloalkyl)alkyl groups, consisting of 1 to 8 carbon atoms, which may contain one or more double or triple bonds, each of which may be further substituted with one or more substituent(s) independently selected from oxo, hydroxy, halogen, nitro, cyano, C₁₋₆alkoxy, —NH(C₁₋₆alkyl), —N(C₁₋₆alkyl)(C₁₋₆alkyl), —NHC(═O)(C₁₋₆alkyl), —N(C₁₋₆alkyl)C(═O)(C₁₋₆alkyl), and Z;
R_{C} is independently selected at each occurrence from halogen, cyano, halo(C₁₋₆)alkyl, halo(C₁₋₆)alkoxy, hydroxy, amino, and C₁₋₆alkyl substituted with 0-2 R_{D}, C₂₋₆alkenyl substituted with 0-2 R_{D}, C₂₋₆alkynyl substituted with 0-2 R_{D}, C₃₋₇cycloalkyl substituted with 0-2 R_{D}, (C₃₋₇cycloalkyl)C₁₋₄alkyl substituted with 0-2 R_{D}, C₁₋₆alkoxy substituted with 0-2 R_{D}, —NH(C₁₋₆alkyl) substituted with 0-2 R_{D}, —N(C₁₋₆alkyl)(C₁₋₆alkyl) each C₁₋₄alkyl independently substituted with 0-2 R_{D}, −XRA, and Y, with the proviso that at least one of the positions ortho or para to the point of attachment of Ar to the pyrimidine ring shown in Formula A is substituted;
R_{D} is independently selected at each occurrence the group consisting of halogen, hydroxy, cyano, C₁₋₄alkyl, —O(C₁₋₄alkyl), —NH(C₁₋₄alkyl), —N(C₁₋₄alkyl)(C₁₋₄alkyl), halo(C₁₋₄)alkyl, halo(C₁₋₄)alkoxy, CO(C₁₋₄alkyl), CONH(C₁₋₄alkyl), CON(C₁₋₄alkyl)(C₁₋₄alkyl), −XRA, and Y;
X is independently selected at each occurrence from the group consisting of —CH₂—, —CHR_{B}—, —O—, —C(═O)—, —C(═O)O—, —NH—, —NR_{B}—, —C(═O)NH—, —C(═O)NR_{B}—, —NHC(═O)—, and —NR_{B}C(═O)—;
Y and Z are independently selected at each occurrence from the group consisting of: 3- to 7-membered carbocyclic and heterocyclic groups, which are saturated, unsaturated, or aromatic, which may be further substituted with one or more substituents independently selected from halogen, oxo, hydroxy, amino, C₁₋₄alkyl, —O(C₁₋₄alkyl), —NH(C₁₋₄alkyl), and —N(C₁₋₄alkyl)(C₁₋₄alkyl); and n is0, 1,or 2.

11. A compound or salt according to claim 9, wherein:
Ar is phenyl mono-, di-, or tri-substituted with R_{C}, and R₁ and R₃ are independently selected from the group consisting of hydrogen, halogen, C₁₋₄alkoxy, halo(C₁₋₄)alkyl, halo(C₁₋₄)alkoxy, C₁₋₆alkyl, which C₁₋₆alkyl is unsubstituted or substituted by one to three substituents independently selected from hydroxy, oxo, cyano, C₁₋₄alkoxy, amino, and mono- or di(C₁₋₄)alkylamino, and (C₃₋₇cycloalkyl)C₁₋₄alkyl, which (C₃₋₇cycloalkyl)C₁₋₄alkyl is unsubstituted or substituted by one to three substituents independently selected from hydroxy, oxo, cyano, C₁₋₄alkoxy, amino, and mono- or di(C₁₋₄)alkylamino.

12. A compound or salt according to claim 9, wherein:
Ar is a phenyl group of the formula: 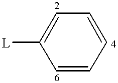
wherein L indicates a bond to the pyrimidine ring in Formula A and the phenyl group is substituted at one, two, or three of positions 2, 4, and 6 positions of the phenyl ring with substituents independently selected from:
i) halogen, cyano, halo(C₁₋₄)alkyl, halo(C₁₋₄)alkoxy, hydroxy, amino, C₁₋₆alkyl, C₁₋₆alkoxy, (C₁₋₄alkoxy)C₁₋₄alkoxy, and mono- or di(C₁₋₄alkyl)amino,
ii) C₁₋₆alkyl and C₁₋₆alkoxy which are further substituted with a 3- to 7-membered carbocyclic and heterocyclic group, which is saturated, unsaturated, or aromatic, which 3- to 7-membered carbocyclic and heterocyclic group may be further substituted with one or more substituents independently selected from halogen, oxo, hydroxy, amino, C₁₋₄alkyl, —O(C₁₋₄alkyl), —NH(C₁₋₄alkyl), and —N(C₁₋₄alkyl)(C₁₋₄alkyl).

13. A compound or salt according to claim 9, wherein:
Ar is phenyl mono-, di-, or tri-substituted with R_{C},
R_{X} and R_{Y}, which may be the same or different, are independently selected at each occurrence from straight, branched, or cyclic alkyl groups, including (cycloalkyl)alkyl groups, consisting of 1 to 8 carbon atoms, which may contain one or more double or triple bonds; and
R₁ and R₃ are independently selected from the group consisting of hydrogen, halogen, C₁₋₄alkoxy, halo(C₁₋₄)alkyl, (halo(C₁₋₄)alkoxy,
C₁₋₆alkyl, which C₁₋₆alkyl is unsubstituted or substituted by one to three substituents independently selected from hydroxy, oxo, cyano, C₁₋₄alkoxy, amino, and mono- or di(C₁₋₄)alkylamino,
(C₃₋₇cycloalkyl)C₁₋₄alkyl, which (C₃₋₇cycloalkyl)C₁₋₄alkyl is unsubstituted or substituted by one to three substituents independently selected from hydroxy, oxo, cyano, C₁₋₄alkoxy, amino, and mono- or di(C₁₋₄)alkylamino.

14. A compound or salt according to claim 3 of the formula: 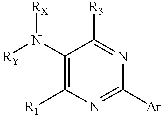
R_{X} and R_{Y} are the same or different and are independently selected from the group consisting of:
hydrogen and C₁-C₆ alkyl; or
NR_{X}R_{Y} represents: 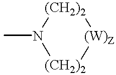
wherein:
z is 0 or 1 ; and
W is chosen from the group consisting of CR_{A}R_{B}, NR_{B}, and O.

15. A compound or salt according to claim 3, of the formula 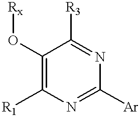
wherein:
R_{X} is chosen from straight, branched, or cyclic alkyl groups, including cycloalkyl(alkyl) groups, having from 1 to 8 carbon atoms, which may contain one or more double or triple bonds, each of which may be further substituted with one or more substituent(s) independently selected from:
(a) hydroxy, halogen, amino, cyano, —O(C₁₋₄alkyl), —NH(C₁₋₄alkyl), and —NH(C₁₋₄alkyl) (C₁₋₄alkyl), and
(b) 3- to 7-membered carbocyclic and heterocyclic groups, which are saturated, unsaturated, or aromatic, which may be substituted with one or more substituents selected from halogen, halo(C₁₋₄)alkyl, oxo, hydroxy, amino, C₁₋₄alkyl, —O(C₁₋₄alkyl), —NH(C₁₋₄alkyl), —N(C₁₋₄alkyl)(C₁₋₄alkyl), wherein said 3- to 7-membered heterocyclic groups contain one or more heteroatom(s) selected from N, O, and S, with the point of attachment being either carbon or nitrogen.

16. A compound or salt according to claim 15 wherein:
R₁ and R₃ are independently selected from the group consisting of hydrogen, halogen, C₁₋₄alkyl, C₁₋₄alkoxy, and halo(C₁₋₄)alkyl.

17. A compound or salt according to claim 3 of Formula B: 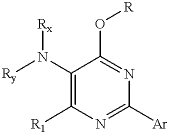
Ar is phenyl mono-, di-, or tri-substituted with R_{C};
R is selected from straight, branched, or cyclic alkyl groups, including (cycloalkyl)alkyl groups, which may contain 1 or more double or triple bonds, and which are optionally substituted by one or more substituents independently chosen from oxo, hydroxy, halogen, cyano, —O(C₁₋₄alkyl), amino, —NH(C₁₋₄alkyl), and —N(C₁₋₄alkyl) (C₁₋₄alkyl);
R₁ is selected from hydrogen, halogen, cyano, C₁₋₄alkyl, (C₃₋₇cycloalkyl)C₁₋₄alkyl, halo(C₁₋₄)alkyl, halo(C₁₋₄)alkoxy, and —O(C₁₋₄alkyl); and
R_{X} and R_{Y} are the same or different and are independently selected from:
a) hydrogen,
b) —(C═O)alkyl_{A}, wherein alkyl_{A} is a straight or branched alkyl group having from 1 to 8 carbon atoms;
c) straight, branched, or cyclic alkyl groups, including cycloalkyl(alkyl) groups, having from 1 to 8 carbon atoms, which may contain one or more double or triple bonds, each of which may be further substituted with one or more substituent(s) independently selected from (i)hydroxy, halogen, amino, cyano, —O(C₁₋₄alkyl), —NH(C₁₋₄alkyl), and —NH(C₁₋₄alkyl)(C₁₋₄alkyl), and (ii)3- to 7-membered carbocyclic and heterocyclic groups, which are saturated, unsaturated, or aromatic, which may be substituted with one or more substituents selected from halogen, halo(C₁₋₄)alkyl, halo(C₁₋₄)alkoxy, oxo, hydroxy, amino, C₁₋₄alkyl, —O(C₁₋₄alkyl), —NH(C₁₋₄alkyl), —N(C₁₋₄alkyl)(C₁₋₄alkyl), and —S(O)ₙ(alkyl), wherein said 3- to 7-membered heterocyclic groups contain one or more heteroatom(s) independently selected from N, O, and S, with the point of attachment being either carbon or nitrogen.

18. A compound or salt according to claim 17, wherein Ar is a phenyl group of the formula: 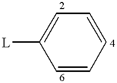
wherein L indicates a bond to the pyrimidine ring in Formula B and the Ar phenyl group is substituted at one, two, or three of positions 2, 4, and 6 with substituents independently selected from:
i) halogen, cyano, halo(C₁₋₄)alkyl, halo(C₁₋₄)alkoxy, hydroxy, amino, C₁₋₆alkyl, C₁₋₆alkoxy, (C₁₋₄alkoxy)C₁₋₄alkoxy, and mono- or di(C₁₋₄alkyl)amino,
ii) C₁₋₆alkyl and C₁₋₆alkoxy which are further substituted with a 3- to 7-membered carbocyclic and heterocyclic group, which is saturated, unsaturated, or aromatic, which 3- to 7-membered carbocyclic and heterocyclic group may be further substituted with one or more substituents independently selected from halogen, oxo, hydroxy, amino, C₁₋₄alkyl, —O(C₁₋₄alkyl), —NH(C₁₋₄alkyl), and —N(C₁₋₄alkyl)(C₁₋₄alkyl).

19. A compound or salt according to claim 17, wherein
Ar is a phenyl group of the formula: 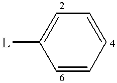
wherein L indicates a bond to the pyrimidine ring in Formula B and the Ar phenyl group is substituted at one, two, or three of positions 2, 4, and 6 with substituents independently selected from:
i) halogen, cyano, halo(C₁₋₄)alkyl, halo(C₁₋₄)alkoxy, hydroxy, amino, C₁₋₆alkyl, C₁₋₆alkoxy, (C₁₋₄alkoxy)C₁₋₄alkoxy, and mono- or di(C₁₋₄alkyl)amino,
ii) C₁₋₆alkyl and C₁₋₆alkoxy which are further substituted with a 3- to 7-membered carbocyclic and heterocyclic group, which is saturated, unsaturated, or aromatic, which 3- to 7-membered carbocyclic and heterocyclic group may be further substituted with one or more substituents independently selected from halogen, oxo, hydroxy, amino, C₁₋₄alkyl, —O(C₁₋₄alkyl), —NH(C₁₋₄alkyl), and —N(C₁₋₄alkyl)(C₁₋₄alkyl);
R_{X} and R_{Y} are the same or different and are independently selected from the group consisting of:
a) hydrogen (with the proviso that R_{X} and R_{Y} are not both hydrogen),
b) —(C═O)alkyl_{A}, wherein alkyl_{A} is a straight or branched alkyl group having from 1 to 8 carbon atoms;
c) straight, branched, or cyclic alkyl groups, including cycloalkyl(alkyl), said straight, branched, or cyclic alkyl groups having from 1 to 8 carbon atoms, and containing zero, one or more double or triple bonds, each of which 1 to 8 carbon atoms may be further substituted with one or more substituent(s) independently selected from hydroxy, halogen, amino, cyano, —O(C₁₋₄alkyl), —NH(C₁₋₄alkyl), and —NH(C₁₋₄alkyl)(C₁₋₄alkyl).

20. A compound or salt according to claim 17, wherein
Ar is a phenyl group of the formula: 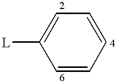
wherein L indicates a bond to the pyrimidine ring in Formula B and the Ar phenyl group is substituted at one, two, or three of positions 2, 4, and 6 with substituents independently selected from:
i) halogen, cyano, halo(C₁₋₄)alkyl, halo(C₁₋₄)alkoxy, hydroxy, amino, C₁₋₆alkyl, C₁₋₆alkoxy, (C₁₋₄alkoxy)C₁₋₄alkoxy, and mono- or di(C₁₋₄alkyl)amino,
ii) C₁₋₆alkyl and C₁₋₆alkoxy which are further substituted with a 3- to 7-membered carbocyclic and heterocyclic group, which is saturated, unsaturated, or aromatic, which 3- to 7-membered carbocyclic and heterocyclic group may be further substituted with one or more substituents independently selected from halogen, oxo, hydroxy, amino, C₁₋₄alkyl, —O(C₁₋₄alkyl), —NH(C₁₋₄alkyl), and —N(C₁₋₄alkyl)(C₁₋₄alkyl); R_{X} and R_{Y} are the same or different and are independently selected from the group consisting of:
a) hydrogen (with the proviso that R_{X} and R_{Y} are not both hydrogen),
b) —(C═O)alkyl_{A}, wherein alkyl_{A} is a straight or branched alkyl group having from 1 to 8 carbon atoms,
c) straight, branched, or cyclic alkyl groups, including cycloalkyl(alkyl) groups, said straight, branched, or cyclic alkyl groups have from 1 to 8 carbon atoms and may contain one or more double or triple bonds.

21. A compound or salt according to claim 17, of the formula: 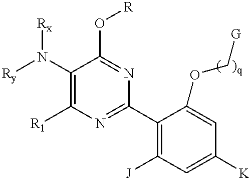
wherein:
q is an integer from 1 to 4;
G is hydrogen, hydroxy, C₁₋₆alkoxy, —NH(C₁₋₆alkyl), —N(C₁₋₆alkyl)(C₁₋₆alkyl), or a 3- to 7-membered carbocyclic or heterocyclic group which is saturated, unsaturated, or aromatic, which is unsubstituted or substituted with one or more substituents independently selected from halogen, halo(C₁₋₄)alkyl, halo(C₁₋₄)alkoxy, oxo, hydroxy, amino, C₁₋₄alkyl, —O(C₁₋₄alkyl), —NH(C₁₋₄alkyl), —N(C₁₋₄alkyl)(C₁₋₄alkyl), and —S(O)ₙ(alkyl), wherein said 3- to 7-membered heterocyclic group contains one or more heteroatom(s) independently selected from N, O, and S, with the point of attachment being either carbon or nitrogen;
J and K are independently selected from halogen, cyano, halo(C₁₋₄)alkyl, halo(C₁₋₄)alkoxy, hydroxy, amino, C₁₋₆alkyl, C₁₋₄alkyl, C₁₋₄alkoxy, (C₁₋₄alkoxy) C₁₋₄alkoxy, and mono- or di(C₁₋₄alkyl)amino.

22. A compound or salt according to claim 17, of the formula: 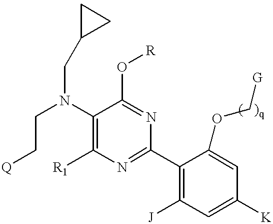
wherein:
Q is hydrogen, C₃₋₇cycloalkyl, pyrrolidinyl, piperidinyl, morpholino, or piperazinyl;
q is an integer from 1 to 4;
G is hydrogen, hydroxy, C₁₋₆alkoxy, —NH(C₁₋₆alkyl), —N(C₁₋₆alkyl)(C₁₋₆alkyl), or a 3- to 7-membered carbocyclic or heterocyclic group, which is saturated, unsaturated, or aromatic, which is unsubstituted or substituted with one or more substituents independently selected from halogen, halo(C₁₋₄)alkyl, halo(C₁₋₄)alkoxy, oxo, hydroxy, amino, C₁₋₄, —O(C₁₋₄alkyl), —NH(C₁₋₄alkyl), —N(C₁₋₄alkyl)(C₁₋₄alkyl), and —S(O)ₙ(alkyl), wherein said 3- to 7-membered heterocyclic group contains one or more heteroatom(s) independently selected from N, O, and S, with the point of attachment being either carbon or nitrogen;
J and K are independently selected from halogen, cyano, halo(C₁₋₄)alkyl, halo(C₁₋₄)alkoxy, hydroxy, amino, C₁₋₆alkyl, C₁₋₄alkyl, C₁₋₄alkoxy, (C₁₋₄alkoxy) C₁₋₄alkoxy, and mono- or di(C₁₋₄alkyl)amino; and
R_{X} and R_{Y} are the same or different and are independently selected from hydrogen (with the proviso that R_{X} and R_{Y} are not both hydrogen) and straight, branched, or cyclic alkyl groups having from 1 to 6 carbon atoms, which alkyl groups may contain one or more double or triple bonds.

23. A compound or salt according to claim 3 of general the formula: 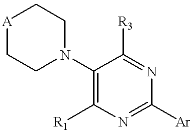
wherein:
A is NH, N(C₁₋₆-alkyl), O, CH₂, or CH(C₁₋₆-alkyl).

24. A compound or salt according to claim 1 wherein, in a standard in vitro CRF receptor binding assay the compound exhibits an IC₅₀ value less than or equal to 1 micromolar.

25. A compound or salt according to claim 1 wherein, in a standard in vitro CRF receptor binding assay the compound exhibits an IC₅₀ value less than or equal to 100 nanomolar.

26. A compound or salt according to claim 1 wherein, in a standard in vitro CRF receptor binding assay the compound exhibits an IC₅₀ value less than or equal to 10 nanomolar.

27. A method for treating an anxiety disorder, a stress-related disorder, or an eating disorder, comprising administering to a patient in need of such treatment a therapeutically effective amount of a compound or salt according to claim 1.

28. A method for treating an depression or bipolar disorder, comprising administering to a patient in need of such treatment a therapeutically effective amount of a compound or salt according to claim 1.

29. A method for treating anorexia nervosa, bulimia nervosa, or obesity, comprising administering to a patient in need of such treatment a therapeutically effective amount of a compound or salt according to claim 1.

30. A compound or salt according to claim 1, wherein in a standard in vitro Na channel functional assay the compound does not show any statistically significant activity at the p<0.05 level of significance.

31. A method for localizing CRF receptors is tissue section samples comprising:
contacting with a sample of tissue a detectably-labeled compound or salt of claim 1 under conditions that permit binding of the compound to CRF receptors within the sample of tissue;
washing the tissue sample to remove unbound compound; and
detecting remaining bound compound, wherein the detection of remaining bound compound is an indication of the presence of CRF receptors in the tissue sample.

32. A method of inhibiting the binding of CRF to the CRF1 Receptor which comprises:
contacting a solution comprising CRF and a compound or salt of claim 1 with a cell expressing a CRF receptor, wherein the compound is present at a concentration sufficient to inhibit CRF binding to IMR32 cells in vitro.

33. The method of claim 32 wherein the cell expressing a CRF receptor is a neuronal cell that is contacted in vivo in an animal, the solution is a body fluid.

34. The method of claim 33 wherein the animal is a human, the cell is a brain cell, and the fluid is cerebrospinal fluid.

35. A pharmaceutical composition comprising a pharmaceutically acceptable carrier and a compound or salt of claim 1.

36. A packaged pharmaceutical composition comprising a pharmaceutical composition of claim 35 in a container and instructions for using the composition to treat a patient suffering from an anxiety disorder, a stress-related disorder, or an eating disorder.

37. A packaged pharmaceutical composition comprising a pharmaceutical composition of claim 35 in a container and instructions for using the composition to treat a patient suffering from depression or bipolar disorder.

38. A packaged pharmaceutical composition comprising a pharmaceutical composition of claim 35 in a container and instructions for using the composition to treat a patient suffering from anorexia nervosa, bulimia nervosa, or obesity.

39. A compound according to claim 1, which is [2-(2,4-dimethoxyphenyl)-4-methoxy-6-methylpyrimidin-5-yl]dipropylamine.

40. A compound according to claim 1, which is [2-(2-chlorophenyl)-4-methoxy-6-methylpyrimidin -5-yl]dipropylamine.

41. A compound according to claim 1, which is [2-(2,4-dichlorophenyl)-4-methoxy-6-methylpyrimidin -5-yl]dipropylamine.

42. A compound according to claim 1, which is [2-(2-methoxy-4-chlorophenyl)-4-methoxy-6-methylpyrimidin -5-yl]dipropylamine.

43. A compound according to claim 1, which is [2-(2-methoxy-4-isopropylphenyl)-4-methoxy-6-methylpyrimidin -5-yl]dipropylamine.

44. A compound according to claim 1, which is [2-(2,4-dimethoxyphenyl)-4-methoxy-6-methyl pyrimidin-5 -yl]dipropyl amine.

45. A compound according to claim 1, which is [4-methoxy-2-(6-m ethoxy-2,4-dimethylphenyl)- 6-methylpyrimi din-5-yl]dipropylamine.

46. A compound according to claim 1, which is [2-(2-methoxy-4,6-dimethylphenyl)-4-methoxy-6-ethyl pyrimidin-5-yl]dipropylamine.

47. A compound according to claim 1, which is [2-(2,4,6-trimethylphenyl)-4-methoxy-6-methyl pyrimidin-5 -yl]dipropylamine.

48. A compound according to claim 1, which is [2-(2,4,6-trimethylphenyl)-4-methoxy-6-ethyl pyrimidin-5-yl]dipropylamine.

49. A compound according to claim 1, which is [2-(2-methoxy-4,6-dimethylphenyl)-4-ethoxy-6-methyl pyrimidin-5-yl]dipropylamine.

50. A compound according to claim 1, which is [2-(2-methoxy-4,6-dimethylphenyl)-4-(2-fluoroethoxy)-6-methyl pyrimidin-5 -yl]dipropylamine.

51. A compound according to claim 1, which is [2-(2-methoxy-4,6-dimethylphenyl)-4isopropoxy-6-methyl pyrimidin-5-yl]dipropylamine.

52. A compound according to claim 1, which is [2-(2-methoxy-4,6-dimethylphcnyl)-4-methoxy-6-fluoromethyl pyrimidin-5-yl]dipropylamine.

53. A compound according to claim 1, which is [2-(2-methoxy-4,6-dimethylphenyl)-4-methoxy-6-difluoromethyl pyrimidin-5-yl]dipropylamine.

54. A compound according to claim 1, which is 1 [5-(dipropylamino)-6-methoxy-2-(2-methoxy-4,6-dimethylphenyl)-pyrimidin-4-yl]-ethan-1-ol.

55. A compound according to claim 1, which is 1-[5-(dipropylamino)-6-methoxy-2-(2-methoxy-4,6-dimethylphenyl)-pyrimidin-4-yl]-propan-2-ol.

56. A compound according to claim 1, which is [4-(2-Cyclopropyl-2-fluoro-ethyl)-6-methoxy-2-(2-methoxy-4,6-dimethyl-phenyl)-pyrimidin-5-yl]-dipropyl-amine.

57. A compound according to claim 1, which is [4-(2-Cyclopropyl-2-hydroxy-ethyl)-6-methoxy-2-(2-methoxy-4,6-dimethyl-phenyl)-pyrimidin-5-yl]-dipropyl-amine.

58. A compound according to claim 1, which is 1-[5-Dipropylamino-6-methoxy-2-(2-methoxy-4,6-dimethyl-phenyl)-pyrimidin-4-ylmethyl]-eyelobutanol.

59. A compound according to claim 1, which is (Cyclopropylmethyl)[4-methoxy-2-(6-methoxy-2,4-dimethylphenyl)-6-methylpyrimidin-5-yl]propylamine.

60. A compound according to claim 1, which is Cyclopropylmethyl-[2-(2-ethoxy-4,6-dimethylphenyl)-4-methoxy-6-methyl pyrimidin-5-yl]propyl-amine.

61. A compound according to claim 1, which is Cyclopropylmethyl[2-(2-propoxy-4,6-dimethylphenyl)-4-methoxy-6-methylpyrimidin-5-yl]dipropylamine.

62. A compound according to claim 1, which is Cyclopropylmethyl[2-(2-isopropoxy-4,6-dimethylphenyl)-4-methoxy-6-methylpyrimidin-5-yl]dipropylamine.

63. A compound according to claim 1, which is Cyclopropylmethyl[2-(2-ethoxymethoxy-4,6-dimethylphenyl)-4-m ethoxy-6-methylpyrimidin-5-yl]dipropylamine.

64. A compound according to claim 1, which is [2-(dimethylamino)ethyl](cyclopropylmethyl)[6-methoxy-2-(6-methoxy-2,4-dimethylphenyl)- 4-methylpyrimidin-5-yl]amine.

65. A compound according to claim 1, which is Cyclopropylmethyl-[4-methoxy-2-(2-methoxy-4,6-dimethyl-phenyl)-6-methyl-pyrimidin-5-yl]-(2-pyrrolidin-1-yl-ethyl)-amine.

66. A compound according to claim 1, which is Cyclopropylmethyl-[4-methoxy-2-(2-methoxy-4,6-dimethyl-phenyl)-6-methyl-pyrimidin-5-yl]-(2-morpholin-1-yl-ethyl)-amine.

67. Cyclopropylmethyl-(2-methoxy-ethyl)-[4-methoxy-2-(2-methoxy-4,6-dimethyl-phenyl)-6-methyl-pyrimidin-5-yl]-amine.

68. A compound according to claim 1, which is Cyclopropylmethyl-[4-methoxy-2-(2-methoxy-4,6-dimethyl-phenyl)-6-methyl-pyrimidin-5-yl]-(2-piperidin-1-yl-ethyl)-amine.
